(19) United States
(12) Patent Application Publication
(10) Pub. No.: US 2023/0203182 A1
(43) Pub. Date: Jun. 29, 2023

(54) Composition and Use of Engineered Monoclonal Antibodies Refractory to Tumor Immuno-Suppressive Factors

(71) Applicant: Navrogen, Inc., Glen Mills, PA (US)

(72) Inventors: Nicholas C. Nicolaides, Glen Mills, PA (US); Luigi Grasso, Bryn Mawr, PA (US); James Bradford Kline, Morgantown, PA (US)

(21) Appl. No.: 17/789,746

(22) PCT Filed: Dec. 29, 2020
(86) PCT No.: PCT/US2020/067345

§ 371 Date: Jun. 28, 2022

Related U.S. Application Data
(60) Provisional application No. 62/955,477, filed on Dec. 31, 2019.

(51) Int. Cl. C07K 16/28; C07K 14/705; C12N 5/09; G01N 33/50

(52) U.S. Cl. CPC C07K 16/2887; C07K 14/705; C12N 5/0693; G01N 33/5011; A61K 38/00; C07K 2317/31

(57) ABSTRACT

The CA125/MUC16 protein has been found to be a suppressor of humoral immunity, in particular, antibody-mediated humoral immunity mediated through the direct binding to a subset of antibodies. Antibody variants can be generated that have reduced or eliminated CA125 binding yet retain the antigen specificity of the parental antibody. These can be used in treating patients with elevated CA125. CA 125-refractory antibodies are developed and used for treatment. Additionally, proteins that enhance humoral immunity in the presence of CA125 can be used to counter the suppression of humoral immunity.

### TECHNICAL FIELD OF THE INVENTION

This invention relates to the area of humoral immunity and humoral immuno-oncology. In particular, it relates to methods and compositions of agents that can overcome the immuno-suppressive effects of the CA125/MUC16 protein to improve antibody-based therapeutic efficacy in inhibiting cancer growth and other humoral immuno-suppressed diseases.

### BACKGROUND OF THE INVENTION

Humoral immunity is a major mechanism by which vertebrate host organisms surveil and defend against dysregulated and transformed host cells. In cancer biology, immune checkpoint inhibitors that can overcome suppressed cellular-mediated immunity have demonstrated robust effects in unleashing activated CD8+ T-cell killing against subsets of tumors (Hodi FS, et al. N Engl J Med 363:711-723, 2010). Several commercially approved therapeutic antibodies have been reported to exhibit their tumor-killing effects through humoral-mediated antibody dependent cellular cytotoxicity (ADCC) and complement dependent cytotoxicity (CDC) (DiLillo DJ, Ravetech JV, Cancer Immunol Res 3:704-713, 2015; Ruck T, et al. Int J Mol Sci. 16:16414-16439, 2015; Pelaia C, et al. Biomed Res Int 4839230:1-9, 2018). Recent translational findings have shown that tumors produce factors that can also suppress humoral immune pathways that in turn suppress the tumor-killing effects via ADCC and CDC (Vergote I, et al. J Clin Oncol 34:2271-2278; Kline JB, et al. J Clin Oncol 5:15, 2018; Wang W et al. Cytogenet Genome Res 152:169-179, 2017; Kline JB et al. Eur J Immunol. 48:1872-1882, 2018).

The antibody-mediated humoral immune response is governed by the coordination of antibody-cell surface antigen engagement that in turn positions the antibody on the antigen epitope at a certain proximity to the cell surface. Once bound, antibodies may engage with Fc-gamma activating receptors FCGR3A and FCGR2A on Natural Killer (NK) or dendritic/myeloid/monocytic cells, respectively, (any cell that participates in ADCC is referred to here as an “immune-effector cell”) to initiate ADCC as well as engage with the C1q complement initiating protein to elicit target cell death of antibody-bound cells via the classical complement CDC pathway (Reuschenbach M, et al. Cancer Immunol Immunother 58:1535-1544, 2009). These mechanisms have been shown to be key in the anti-tumor effects of therapeutic antibodies such as, but not limited to, rituximab, trastuzumab, cetuximab, alemtuzumab as well as a number of experimental antibodies (Zhou X, et al. Oncologist 13:954-966, 2008; Hsu YF, et al. Mol Cancer 9:-8, 2010; Spiridon CI, et al. Clin Cancer Res 8:1720-1730, 2002; Kline JB, et al. Eur J Immunol 48:1872-1882, 2018). Similarly, humoral immune responses have been shown to occur within a patient’s own immune system to target dysregulated or transformed cells in response to vaccines and those with indolent disease yielding antibodies predominantly of the IgM class with anti-proliferative as well as immune-mediated target cell killing activities (Staff C, et al. J Clin Immunol 32:855-865; Branden S, et al. Cancer Res 63:7995-8005, 2003). Yet, while many cancer patients have been found to produce autoantibodies to tumor-expressed antigens, their presence is not sufficient for controlling tumor growth likely due to overall antibody levels and/or humoral immuno-suppression mechanisms deployed by tumor cells.

Several reports have found that the tumor produced protein MUC16/CA125 may suppress humoral immune responses through direct binding to a subset of IgG1, IgG3 and IgM type antibodies that in turn perturb the structure of the Fc region making it less effective for IgG1 and IgG3 type antibodies to engage with Fc-gamma activating receptors FCGR2A (also referred to as CD32a) and FCGR3A (also referred to as CD16a) on immune-effector cells and/or for all three antibody classes to engage with complement-mediating proteins, including C1q (Pantankar MS, et.al. Gyncol Oncol 99:704-713, 2005; Kline JB, et al. OncoTarget 8:52045-52060, 2017; Kline JB, et al. J. Clin. Oncol. 5:15, 2018; Wang W, et al. Cytogenet Genome Res 152:169-179, 2017; Kline JB, et al. Eur J Immunol. 48:1872-1882, 2018). Clinical studies of anti-cancer antibodies that rely on immune-effector mechanisms for their pharmacologic activity have shown an association of elevated serum CA125 levels with reduced clinical outcomes. In particular, this has been reported in clinical studies of the commercially approved rituximab antibody in patients with Hodgkin’s Lymphoma and Non-Hodgkin’s Lymphoma. Patients with follicular lymphoma that where treated with rituximab plus chemotherapy [CHOP (cyclophosphamide, doxorubicin (hydroxydaunomycin), vincristine (Oncovin®), and prednisolone)] had a 31.4% improvement in 5-year progression free survival (PFS) when CA125 levels were in the normal range (Prochazka V, et al. Int J Hematol 96:58-64, 2012) in contrast to those above the normal range that had a statistically significant worse clinical outcome. There is a continuing need in the art to develop tools and agents that may overcome the humoral immuno-suppression mediated by CA125/MUC16 (referred to herein as CA125) and similar tumor-produced or -induced proteins in cancer patients as well as other diseases in which humoral immuno-suppression is active by the presence of such proteins.

### SUMMARY OF THE INVENTION

According to one aspect of the invention, a method of treating a cancer patient or a patient with an inflammatory disease is provided. A human C3B or C4B complement protein is administered to the cancer patient or the patient with an inflammatory disease. The administered protein enhances the effect of certain therapeutic antibodies, such as rituximab or the effect of autoantibodies. The complement protein may be, e.g., a full-length human complement proprotein C3B or C4B, a naturally occurring, proteolytic fragment of human complement protein C3B or C4B, or a portion of human complement protein C3B or C4B that is capable of binding IgG.

According to another aspect of the invention a protein or polypeptide comprising the amino acid residue sequence of SEQ ID NO: 3 is provided. One, two, or three amino acid residues in said sequence are substituted with a different amino acid residue than shown in SEQ ID NO: 3. The one, two, or three amino acid substitutions reduce or eliminate the binding of the isolated protein or polypeptide to an immuno-suppressive protein, relative to the binding of the isolated protein or polypeptide without the one, two or three amino acid substitutions.

Another aspect of the invention is a method to treat a patient with a disease who expresses an elevated level of CA125 compared to a population of healthy humans. A protein or polypeptide is administered to the patient. The protein or polypeptide comprises the amino acid residue sequence of SEQ ID NO: 3. One, two, or three amino acid residues in the sequence are substituted with a different amino acid residue than shown in SEQ ID NO: 3.

The one, two, or three amino acid substitutions reduce or eliminate the binding of the isolated protein or polypeptide to an immuno-suppressive protein, relative to the binding of the isolated protein or polypeptide without the one, two or three amino acid substitutions.

Still another aspect of the invention is a method for monitoring a tumor expressing CA125 for binding to an antibody. A body fluid sample isolated from the patient is contacted with human C3B or C4B complement protein and with a protein or polypeptide comprising the amino acid residue sequence of SEQ ID NO: 3. One, two, or three amino acid residues in the sequence are substituted with a different amino acid residue than shown in SEQ ID NO: 3. The one, two, or three amino acid substitutions reduce or eliminate the binding of the isolated protein or polypeptide to an immuno-suppressive protein, relative to the isolated protein or polypeptide without the one, two or three amino acid substitutions. CA125 bound to the protein or polypeptide is detected.

Yet another aspect of the invention is a combination comprising an anti-CD20 antibody and a human complement protein C3B or C4B. The form of the human complement protein may be, e.g., a full-length human complement proprotein C3B or C4B, a naturally occurring, proteolytic fragment of human complement protein C3B or C4B, or a portion of human complement protein C3B or C4B that is capable of binding IgG. Combinations can be formed in a vessel prior to administration. Alternatively, each component can be separately administered within a period of time so that the combination forms in vivo. Typically the two are administered within one week, 3 days, 2 days, 24 hours, 12 hours, 6 hours, 3 hours, 1 hour.

One aspect of the invention is a recombinant protein comprising full length or a fragment containing the antibody binding sequences of the human C3B (SEQ ID NO: 1) or C4B (SEQ ID NO: 2) complement proteins which can be added/used in combination with a CA125-imunosuppressed therapeutic antibody to overcome its immunosuppression (referred herein as enhancer proteins).

Another aspect of the invention is a nucleic acid vector encoding a genetically modified rituximab protein in which one or more codons have been modified that in turn reduces CA125 binding to the mature antibody or antibody antigen-binding fragment or Fab domain. In particular are modifications to the region within the antibody heavy chain (SEQ ID NO: 3). These include but are not limited to codons that alter amino acids

AVYYCARSTYYGGDWYFNVWGAGTTVTVSAASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSCDKTHTCPP
(SEQ ID NO: 3)

either as single or a combination of amino acid substitutions. Nucleic acid vectors may encode the genetically modified rituximab protein in proper position relative to other elements, such that the genetically modified protein is expressed from the nucleic acid when it is transfected into cells.

Another aspect of the invention is a nucleic acid vector (SEQ ID NO: 4) encoding a full length rituximab heavy chain with amino acid changes of N to D at codon 109, P to S at codon 131, and S to Y at codon 136 (SEQ ID NO: 5). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 7) encoding a full length rituximab heavy chain with amino acid change of N to D at codon 109 (SEQ ID NO: 8). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 9) encoding a full length rituximab heavy chain with amino acid change of P to S at codon 131 (SEQ ID NO: 10). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 11) encoding a full length rituximab heavy chain with amino acid change of S to Y at codon 136 (SEQ ID NO: 12). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 13) encoding a full length rituximab heavy chain with amino acid changes of N to D at codon 109, and P to S at codon 131 (SEQ ID NO: 14). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 15) encoding a full length rituximab heavy chain with amino acid changes of N to D at codon 109, and S to Y at codon 136 (SEQ ID NO: 16). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 17) encoding a full length rituximab heavy chain with amino acid changes of P to S at 131 and S to Y at 136 (SEQ ID NO: 18). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 19) encoding a full length rituximab heavy chain with amino acid changes of Y to C at codon 107, and A to T at codon 122 (SEQ ID NO: 20). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 21) encoding a full length rituximab heavy chain with amino acid change of Y to C at codon 107 (SEQ ID NO: 22). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 23) encoding a full length rituximab heavy chain with amino acid change of A to T at codon 122 (SEQ ID NO: 24). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 25) encoding a full length rituximab heavy chain with amino acid changes of F to L at codon 108, and F to Y at codon 130 (SEQ ID NO: 26). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 27) encoding a full length rituximab heavy chain with amino acid change of F to L at codon 108 (SEQ ID NO: 28). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 29) encoding a full length rituximab heavy chain with amino acid change of F to Y at codon 130 (SEQ ID NO: 30). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 31) encoding a full length rituximab heavy chain with amino acid changes of G to C at codon 103, P to S at codon 134, S to T at codon 169, and S to R at codon 196 (SEQ ID NO: 32). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 33) encoding a full length rituximab heavy chain with amino acid change of G to C at codon 103 (SEQ ID NO: 34). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 35) encoding a full length rituximab heavy chain with amino acid change of P to S at codon 134 (SEQ ID NO: 36). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 37) encoding a full length rituximab heavy chain with amino acid change of Y to F at codon 102 (SEQ ID NO: 38). The heavy chain can be co-expressed with the rituximab light chain (SEQ ID NO: 6) in a recombinant cell host to yield a fully functioning CD20-binding antibody.

Another aspect of the invention is a nucleic acid vector (SEQ ID NO: 39) encoding a full length rituximab heavy chain with amino acid change of L to Q at codon 167 (SEQ ID NO: 40). The heavy chain can be co-expressed with the rituximab light chain (SEQ ID NO: 6) in a recombinant cell host to yield a fully functioning CD20-binding antibody.

Another aspect of the invention is a method to treat a patient with a disease who expresses an elevated level of CA125 compared to a population of healthy humans. A variant rituximab antibody (referred to herein as RTX-MT) is administered to the patient. The antibody binds to CD20+ target cells and elicits a humoral immune response, such as antibody dependent cellular cytotoxicity (ADCC) or complement dependent cytotoxicity (CDC), to kill the target cells.

Another aspect of the invention is a human cancer cell line that expresses human proteins CA125 and CD20. The human cancer cell line may be a human ovarian cancer cell line that does not naturally express CD20. It may be made by transducing cells of cell line OVCAR3 with an expression vector encoding CD20. Other human cancer cell lines can be used to make such modified cell lines for use in identifying humoral immuno-suppressed antibodies.

In yet another aspect, the invention is a method of screening candidate antibodies that bind to CD20. A candidate antibody is contacted with a human cancer cell line that expresses human proteins CA125 and CD20. Antibody dependent cellular cytotoxicity (ADCC) or complement dependent cytotoxicity (CDC) of the human cancer cell line initiated by the candidate antibody is then determined.

Still another aspect of the invention is a method for testing and treating a tumor expressing CA125 in a patient. A body fluid sample isolated from the patient (component (a)) is contacted with an antibody comprising the amino acid sequence of SEQ ID NO: 3 (component (b)). Component (a) is also contacted with an immuno-suppression refractory protein or polypeptide comprising the amino acid residue sequence of SEQ ID NO: 3, wherein one, two, or three amino acid residues in said sequence are substituted with an amino acid residue different than shown in SEQ ID NO: 3 (component c), wherein the one, two, or three substituted amino acid residues reduce or eliminate binding of the immuno-suppression refractory protein or polypeptide to an immuno-suppressive protein, relative to the immuno-suppression refractory protein or polypeptide without the one, two, or three amino acid substitutions. When CA125 in the body fluid sample (component (a)) is determined to bind to component (b) but not component (c), the patient is treated with component (c). When CA125 in the body fluid sample is determined to bind to component (b) and/or component (c), the patient is treated with full-length complement proprotein C3B or C4B, a naturally occurring, proteolytic fragment of complement proprotein C3B or C4B that is capable of binding IgG, or a portion of complement proprotein C3B or C4B that is capable of binding IgG.

These and other aspects of the invention, which will be apparent to those skilled in the art upon reading the specification, provide the art with methods and compositions for use in improving anti-CD20 antibody-mediated humoral immune responses in CA125 immuno-suppressed diseases, including cancer as well as non-oncologic diseases and in non-CA125 elevated disease types.

### BRIEF DESCRIPTION OF THE DRAWINGS

FIGS. 1A-1D. Screening method to identify potential proteins that can block CA125 binding to affected rituximab antibody (Ab). (FIG. 1A) Schematic diagram of the method to screen for potential enhancer proteins capable of overcoming the immuno-suppressive effects of CA125 on affected antibodies. (FIG. 1B) Identification of enhancer proteins to block CA125 binding and overcome CA125 immuno-suppression of rituximab effector activity. Briefly, 96-well ELISA plates were coated with 1-5 µg/mL rituximab with or without 15 KU/mL of human CA125 or 1-10 µg/mL of human serum albumin (HSA) and probed with 1-2.5 µg/mL biotinylated human C1q protein as previously described (Kline JB, et al. Eur J Immunol. 48:1872-1882, 2018). As shown, CA125 reduced C1q binding to rituximab in contrast to when incubated with rituximab alone or in the presence of HSA. Next, a number of proteins that have been previously reported to bind IgG1 antibodies were used to determine if any could overcome CA125 suppression of C1q binding. As shown in FIG. 1C, the human complement protein C4B (SEQ ID NO: 2) was able to block the inhibitory effect of CA125 in contrast to others tested (not shown). Similarly, C3B (SEQ ID NO: 1) had similar effects. To determine if this effect could restore immune-effector function, the C4B protein was used in complement dependent cytotoxicity (CDC) assays using rituximab and the Daudi cells to test C4B activity in CDC assays as previously described (Kline JB et al. Eur J Immunol. 48: 1872-1882, 2018). Briefly, 5,000 Daudi cells were incubated with 2.5 µg/mL rituximab alone, 15 KU/mL CA125 or 10 µg/mL HSA, with or without 5 µg/mL C4B. Next, 1000 units of rabbit complement (Bio-Rad) was added to each well and cultures were incubated for 1.5 hours then quantified for cell survival using Cell Titer-Glo (Promega) and quantified on a Varioskan luminescent plate reader according to the manufacturer’s recommendation. Signal intensity refers to living cells after CDC killing. (FIG. 1C) CA125 had a significant suppression in rituximab-mediated CDC activity (P < 0.00015). The addition of C4B was able to significantly overcome CA125 suppression (P = 0.00004). Similar effects were observed with C3B (SEQ ID NO; 1) protein. These data provide evidence that the C3B and/or C4B proteins can be used to reverse CA125 immuno-suppressive effects and act as enhancer proteins. (FIG. 1D) Competition of C3B to CA125 binding to RTX-WT is shown; methods are the same as described for FIG. 1B.

FIGS. 2A-2B. C3B/C4B enhancer proteins overcome CA125 immuno-suppression by blocking CA125 binding to rituximab. To uncover the mechanism by which C4B rescues rituximab immune suppression by CA125, ELISA assays were used to monitor CA125-rituximab binding. Briefly, 96-well ELISA plates were coated with 15 KU/mL of soluble CA125 or human serum albumin (HSA) used as a negative control and tested for CA125 binding by probing with 2.5 µg/mL of biotinylated rituximab with or without 5 µg/mL C4B. After probing, wells were washed, secondarily probed with streptavidin-HRP (horse radish peroxidase; Jackson Immunologicals) and quantified using colorimetric TMB (3,3′,5,5′ tetramethylbenzidine) substrate (Pierce). Reactions were stopped using 0.1 N H₂SO₄ and wells quantified using a Varioskan plate reader at 450 nm. As shown in FIG. 2A, the C4B could significantly block rituximab binding to CA125 (P = 0.000017). Experiments represent a minimum of triplicate wells. The C4B protein has been shown to bind to the region between complementarity determine region 3 (CDR3) and framework 4 (FW4) of IgG1-type antibodies, suggesting its competition for CA125 binding may be within this region. To determine if CA125 binding occurs on the light, heavy or both immunoglobulin chains, we generated chimeric rituximab (RTX) antibodies using the light and heavy chain from the CA125 unaffected antibody pertuzumab (PTZ) (Kline JB, et al. OncoTarget 8:52045-52060) and then tested each for CA125 binding. As shown in FIG. 2B, the rituximab heavy chain (RTX-hc/PTZ-lc) chimera could still significantly bind CA125 (P < 0.0009) suggesting that CA125 binds to the rituximab heavy chain within the CDR3-FW4 region. No CA125 binding was observed with the rituximab light chain (PTZ-hc/RTX-lc) chimera. Using similar methods as for C4B, the C3B protein (SEQ ID NO: 1) competed with rituximab binding to CA125 (P < 0.0001) and appeared to be specific as the Fab binding protein L did not competitively inhibit (see FIG. 1D) supporting the finding that CA125 binding is located within the rituximab Fab domain and that C3B /C4B proteins can be used to reverse CA125 immuno-suppressive effects and act as an enhancer proteins.

FIGS. 3A-3B. Generation of rituximab variants with reduced CA125 binding. (FIG. 3A) To determine if one or more residues within the CDR3-FW4 and CH1 regions (SEQ ID NO: 3) were required for CA125 binding, we generated a library of rituximab heavy chain variants between the residues encoding CDR3-FW4 via random mutagenesis of PCR products, using the primers (SEQ ID NOS: 48 and 49) which are complementary to the nucleotide sequence of the CDR3-FW4 region (SEQ ID NO: 50). Recombinant heavy chains were then co-transfected with the rituximab light chain (SEQ ID NO: 6) and expressed transiently in HEK293 cells in 96-well plates. Culture supernatants were screened for antibody production via ELISA using a mouse anti-human IgG-HRP antibody as probe. Positive clone supernatants were then tested for CD20 binding using an ELISA in which the rituximab-binding peptide Rp1-L (Favoino et al., Int J Mol Sci. 20:1920, 2019) was used to coat wells and probed with rituximab mutants (RTX-MT) and secondarily probed with a mouse anti-human IgG-HRP and quantified using TMB substrate as described above. Clones that bound CD20 as well as or better than the parental rituximab antibody (RTX-WT) were then tested for CA125 binding via ELISA as described above. A number of clones were identified that bound CD20 and appeared to have reduced CA125 binding. The sequences of these clones were aligned to identify commonly mutated amino acids (FIG. 3B). A subset of these clones were then expanded to produce purified antibody and retested for CD20 and CA125 binding.

FIG. 4. Mutant rituximab (RTX-MT) clones with the strongest CD20 binding and most reduced CA125 binding identified from the randomized library primary screens were selected and further analyzed. Each of these antibodies were biotinylated and retested for CD20 and CA125 binding via ELISA. Briefly, antibodies were biotinylated using the EZ-Link™ Sulfo-NHS-Biotin (ThermoScientific) following the manufacturer’s instructions. Biotinylated antibodies where quantified by Nanodrop and validated for signal intensity using an anti-IgG ELISA capture assay to measure the signal intensity for each antibody. To retest for CD20 and CA125 binding, 96-well plates were coated with 10 µg/mL of the rituximab-binding peptide or 15 KU/mL of CA125 and probed using each biotinylated RTX-MT antibody or parental antibody (RTX-MT) in triplicate. Wells were washed and then secondarily probed with streptavidin-HRP and quantified using TMB substrate as described above on a Varioskan plate reader at 450 nm. The ratio of CD20 to CA125 binding was compared for the parental and RTX-MT clones and percent decrease in CA125 binding was calculated. Any signal greater than a 20% decrease in CA125 was considered positive. Using these criteria 5 clones, all expressing the wild type rituximab light chain (SEQ ID NO: 6), were selected for further analysis. Clone 148 (SEQ ID NO: 37 and 38); Clone 164 (SEQ ID NO: 31 and 32); Clone 166 (SEQ ID NO: 4 and 5); Clone 198 (SEQ ID NO: 19 and 20) and Clone 199 (SEQ ID NO: 25 and 26).

FIG. 5. Selected RTX-MT antibodies as described in FIG. 4 were tested in antibody dependent cellular cytotoxicity (ADCC) and CDC functional screens. Human CD20-expressing Daudi cells were used for both assays. CDC was carried out as described above. ADCC activity was measured using methods as previously described (Kline JB, et al. OncoTarget 8:52045-52060). As shown, CA125 had a significant suppression of CDC activity on parental antibody (32% reduction; P = 0.001) while all RTX-MT clones tested here were found to be more refractory to CA125 immuno-suppression (≤ 15% CDC suppression; P < 0.01). A similar effect was observed for ADCC. In particular, clones 164, 166 and 198 were approximately 3to 4 times more refractory to CA125 immuno-suppression than the parental antibody while the other clones were also found to be more refractory but to a lesser extent. In all cases, these clones had multiple mutations in the affected domain. To determine if one or two amino acids were sufficient to overcome CA125 immuno-suppression, single or double mutant clones were generated from each clone and retested for CA125 binding and immune-effector function.

FIGS. 6A-6B. Single and double mutations within the affected domain are sufficient to reduce CA125 binding and generate CA125 refractory rituximab antibodies. Single and double RTX-MT antibodies derived from clones 148, 164, 166, 198 and 199 were used in CD20 and CA125 ELISA binding assays (FIG. 6A) as well as CD16a and C1q binding assays to determine if a single or double mutation was sufficient to reduce CA125 binding and immuno-suppression. As shown in FIG. 6A, clone 112-134 (amino acid changes of N to D at codon 109 and P to S at codon 131, SEQ ID NOs: 13 and 14), clone 133 (amino acid change of F to Y at codon 130, SEQ ID NOs: 29 and 30), clone 106 (amino acid change of G to C at codon 103, SEQ ID NOs: 33 and 34), and clone 137 (amino acid change of P to S at codon 134, SEQ ID NOs: 35 and 36) had the most significantly reduced binding to CA125 (P < 0.0055), and they retained CD20 binding like the parental antibody. The two most refractory clones, 112-134 and 133, were used in CD16a-biotin and C1q-biotin binding assays. As shown in FIG. 6B, clones 112-134 and 133 had a 75% and 62.5% improvement in CD16a-biotin binding, respectively, and both had a 70% improvement in C1q-biotin binding as compared to the parental antibody.

FIGS. 7A-7D. Characterization of RTX-MT clone 166 variant mutations. Single or double mutations N109D, P131S, S136Y, N109D+P131S, N109D+S136Y, P131S+S136Y, as well as triple mutant RTX-MT clone 166 (N109D+P131S+S136Y) were compared for Fc receptor binding in the presence of CA125 (FIG. 7A). Data are plotted as a percentage of binding with CA125 versus no CA125. As shown, RTX-MTs carrying at least the N109D mutation had higher binding to CD 16a Fc receptor compared to parent RTX in the presence of CA125. Single mutant RTX-MTs N109D, P131S, and S136Y were compared for C1q binding in the presence of CA125 (FIG. 7B). Data are plotted as percent of binding with CA125 versus no CA125. RTX-MT N109D was found to be the least affected by CA125 retaining ~90% of C1q binding compared to < 65% for parental rituximab (RTX). Antibody binding to CD20 rituximab-binding peptide was measured using an ELISA assay to determine if CD20 binding was affected by the N109D mutation (FIG. 7C). As shown, RTX-MT N109D CD20 binding was comparable to the parental rituximab (RTX) (P > 0.21). ELISA analysis of CA125 binding to RTX-MT N109D showed that it was significantly reduced as compared to parental rituximab (RTX) (50% reduction, P < 0.00009) (FIG. 7D). To ensure similar amounts of antibody were used in each experiment, ELISA assays included anti-human IgG coated wells to capture antibody input amounts. As shown in FIGS. 7C and 7D, the amount of each antibody input was similar (line with square markers). In all experiments, pertuzumab (PTZ) was used as a negative control. Representative P values using student’s T-test are * < 0.05, ** < 0.01, *** < 0.001, **** < 0.0001.

FIGS. 8A-8D. Characterization of RTX N109D CD20 binding. Parental rituximab (RTX WT) and RTX N109D were directly conjugated to AlexaFluor 488 dye and used to stain live CD20 expressing (Ramos) and CD20 null (Jurkat) cells at concentrations ranging from 0.78 to 100 nM. FIGS. 8A and 8B show specific binding of both antibodies to CD20-positive cells but not CD20-null cells, respectively. FIGS. 8C and 8D demonstrate the extrapolated of binding affinity (Kd) as the concentration of antibody bound to half the receptor sites at equilibrium using a nonlinear regression analysis of one-site saturation binding.

FIGS. 9A-9B. Comparison of complement-mediated killing (CDC) by RTX N109D versus parental rituximab (RTX). CD20-positive cells expressing CA125 were targeted with each antibody in the presence of complement (FIG. 8A). As shown, RTX N109D has superior CDC activity against CA125-positive cells compared to parental rituximab. To demonstrate that the CDC effect was specific, CD20-positive and CD20-negative cells expressing CA125 were tested for RTX N109D CDC activity (FIG. 9B). As shown, RTX N109D CDC killing was specific to CD20-expressing cells. Representative P values using student’s T-test are * < 0.05, ** < 0.01, *** < 0.001, **** < 0.0001.

FIGS. 10A-10C. Comparison of CA125 immuno-suppression on Fc-gamma receptor CD16a activity by RTX-MT N109D and parental rituximab (RTX). ELISA binding assays show that RTX-MT N109D is refractory (> 90% Fc receptor binding) to CA125 suppressed CD16a receptor binding as compared to parental rituximab (< 60%, P < 0.0001) (FIG. 10A). Jurkat CD16a reporter assays show that RTX-MT N109D is refractory to CA125 suppressed CD16a receptor activation (P < 0.0001) as compared to parental rituximab (FIG. 10B). ADCC assays using primary human PBMCs and Daudi target cells show that RTX MT N109D is refractory to CA125 suppressed ADCC (P < 0.01) (FIG. 10C). Representative P values using student’s T-test are * <0.05, ** <0.01, *** <0.001, **** <0.0001.

### DETAILED DESCRIPTION OF THE INVENTION

We have found agents (referred to as enhancer proteins) that can block the binding of CA125 to rituximab. We used them to map regions within the rituximab antibody that are critical for CA125 binding and its immunosuppressive activity. Moreover, we used genetic engineering within a particular region of the rituximab antibody that is important for CA125 binding to generate modified antibody proteins that are refractory to CA125’s humoral immunosuppressive effect. These can be used for treating CD20⁺ cells in diseases, including cancer, in which CA125 is elevated, as well as in patients whose CA125 levels are within the normal range.

We have modified residues within a region (affected domain) of an IgG1-type antibody that appears to bind CA125; the modified residues make a CA125-immunosuppressed antibody refractory to CA125 immuno-suppression. These CA125-refractory antibodies are potentially capable of overcoming humoral immuno-suppression by the CA125 protein. They can be used for treating cancers as well as other CA125 immuno-suppression diseases. While not wanting to be limited to any particular theory or mechanism of action, applicants believe that the CA125 protein engages with certain residues of certain antibodies and disrupts the antibody-mediated humoral immune responses normally mediated by immune-effector cells and/or complement. These disruptions include suppression of the C1q-antibody (classical antibody-complement) complex and/or antibody binding to Fc-gamma activating receptors on immune-effector cells, such as but not limited to NK and myeloid cells; these disruptions result in the downstream inhibition of complement dependent cytotoxicity (CDC) and antibody dependent cellular cytotoxicity (ADCC), respectively.

The methods described here may be used to develop additional CA125-refractory antibodies (whether CD20-targeting antibodies or not) to overcome humoral immuno-suppression. Modified antibodies that are capable of evading the inhibitory effect(s) of CA125 on humoral immune responses including ADCC and/or CDC that negatively impact the affected parent antibody may be used for preclinical and human testing.

The methods and compositions described here may be divided into at least two categories. In one category, a modified heavy chain containing one or more amino acid changes (referred to as RTX-MT) is tested to determine maximal effectiveness in overcoming CA125 suppression on ADCC and/or CDC as measured using molecular assays that monitor the binding of the Fc-gamma activating receptors CD16a or CD32a or C1q complement protein to RTX-MT antibody as compared to the parental antibody in the presence of CA125. In another category, the C3B (SEQ ID: 1) or C4B proteins (SEQ ID: 2) are tested in combination with parental or RTX-MT antibodies to determine maximal effectiveness in overcoming CA125 suppression on ADCC and/or CDC as determined using cell viability assays. Antibodies can be added to CD20 antigen-positive target cells expressing CA125 or exogenously added CA125 to non-CA125 expressing cells with the C3B or C4B protein and ADCC activity measured by addition of immune-effector cells (NK, dendritic/myeloid/monocytic cells, peripheral blood mononuclear cells or ADCC reporter cell lines). Alternatively, human or rabbit complement can be used to monitor CDC activity. In either assay, cell viability is compared between cells treated with parental vs RTX-MT antibodies with or without the C3B or C4B proteins to determine which RTX-MT and/or combination of C3B or C4B can significantly block CA125-mediated humoral immune suppression. CA125 can be produced from target cells or added exogenously to the cell culture. Cell viability can be determined employing a variety of methods used by those skilled in the art.

For therapeutic applications, the C3B or C4B proteins can be administered in combination with parental RTX-WT or RTX-MT antibody with or without other standard-of-care agents. They can be administered prior, during or after administration of the rituximab antibody.

Compositions can be formed in the course of conducting the methods. They may be combinations of proteins with altered amino acids within the affected domain of RTX (SEQ ID NO: 3) identified from the rituximab heavy chain mutant library screen or other enhancer proteins that can be added to the parental RTX-WT or RTX-MT antibody to enhance immune-effector activity, for example. Any selection of antibodies and/or enhancer protein (i.e., proteins containing antibody binding domains of the C3B and C4B proteins) described here may be formed as a composition, before or after administration.

While a few antibodies that are susceptible to CA125 immuno-suppression are known, the methods to develop compositions by modifying the affected domain of RTX shown in SEQ ID NO: 3 as described here are useful for any antibody whose dynamic structure is altered by CA125, which leads to suppressed humoral immune responses.

The “dynamic structure” of an antibody or protein is the three-dimensional structures of an antibody at a given time point, wherein such time point coincides with the antibody’s engagement with another protein or agent, and the structure of the antibody before this time point has changed into a different structure after this time point in response to the antibody’s engagement with another protein or agent. One can monitor this change in the presence of enhancer proteins such as C3B and C4B. The effect of a protein’s binding to an antibody and affecting its dynamic structure has been reported in the case of hapten binding to the CDR domains of antibodies that in turn allosterically alter their Fc domain, thereby reducing the Fc domain’s engagement with Fc binding proteins including Fc receptors (Janda A, et al. Front Microbiol 7:22, 2016). Previous studies also have shown that the dynamic structure of the (FAB′)₂ domain is affected when an antibody was bound to its antigen (Werner TC, et al. Proc Natl Acad Sci 69:795-799, 1972). Recently, Kline et al reported that while several humanized monoclonal IgG1-type antibodies have similar amino acid structure, there are profound differences in the ability of CA125 to bind them (Kline JB et al. OncoTarget 8:52045-52060, 2017).

In light of growing evidence that tumors utilize various pathways to evade host immune defense and the fact that antibody-based approaches continue to be pursued against various cancer types as well as inflammatory and infectious diseases, it is important to identify agents such as C3B and C4B as well as regions within affected antibodies that can be modified to define methods that can overcome the humoral immuno-suppression of affected antibodies. These agents or modified antibody compositions and methods in turn enable the selection of lead antibodies that may overcome the immune suppression by CA125 and be useful for patient screening. For example, if a test antibody is found to be affected by CA125, one may prescreen patients with CD20-positive cancers to determine if they have elevated CA125. For those patients that do, one can use an optimized RTX-MT that can overcome CA125’s inhibitory effect on humoral responses. Alternatively, one can use an enhancer protein such as C3B or C4B along with the parental RTX-WT or RTX-MT antibody to enhance tumor cell killing.

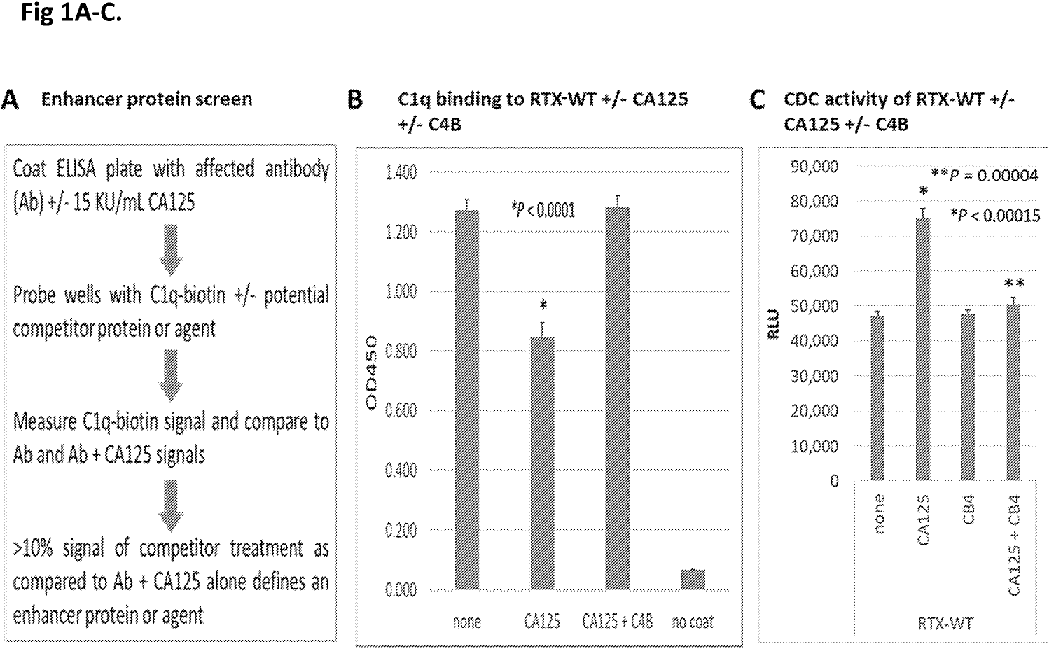

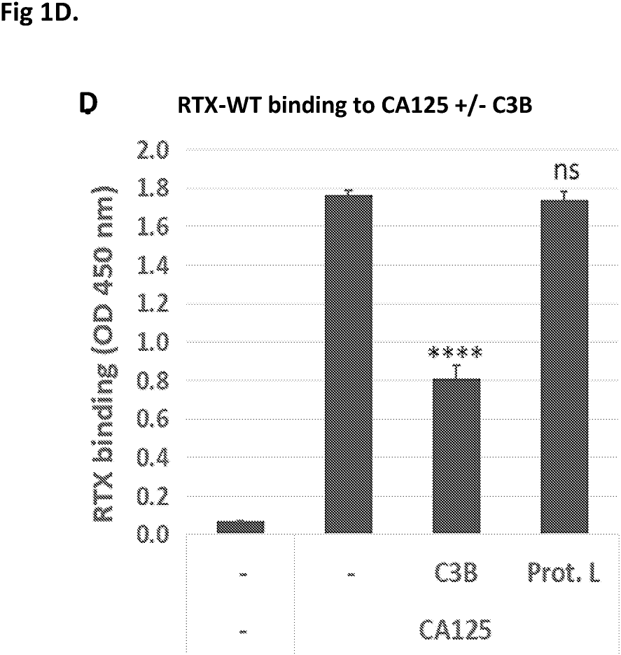

We provide compositions and methods for screening an anti-CD20 antibody’s dynamic structure in the presence of CA125 that can affect antibody dynamic structure and suppress its downstream immune-effector function(s) upon binding to its cell surface target antigen. The methods include a step of making modified heavy chains from the rituximab parental antibody via random mutagenesis of the affected domain contained within SEQ ID NO: 3 and expressing them in a cell line that also expresses the rituximab immunoglobulin light chain (SEQ ID NO: 6) and screening to identify those proteins with reduced CA125 binding or ADCC or CDC immune-effector activity. Additionally, parental rituximab can be screened along with other proteins to identify those proteins or other agents that may enhance its immune-effector activity in the presence of CA125. FIGS. 1A-1D provide an example of combining proteins with rituximab in the presence of CA125 to identify those that can block CA125 binding to the parental antibody and/or enhance its ADCC or CDC activity.

In one embodiment, a human ovarian cancer cell that naturally expresses CA125 is transduced with a human CD20 encoding expression construct to develop a CA125-positive and CD20-positive cell that can be used to screen for CA125 immuno-suppression of CD20 targeting antibodies. Other cancer cell types can be used to make the cell line, particularly cancers derived from epithelial cells, like ovarian cancer cells. Once such cell line is OVCAR.

In some embodiments, the RTX-MT comprises one or more amino acid changes within the affected domain (SEQ ID NO: 3) and is tested for the ability of RTX-MT antibody to overcome CA125 humoral immune suppression of ADCC or CDC.

In one method for measuring the ability of an enhancer protein in combination with the parental or RTX-MT antibody, or an RTX-MT antibody alone, to be effective in overcoming CA125 humoral immuno-suppression, the antibody is tested for direct CA125 binding and/or ability to bind CD16a or C1q proteins in the presence of CA125 via ELISA or other methods known to those skilled in the art.

In some embodiments, functional methods are used to measure the effect of RTX-MT on overcoming humoral immune suppression by CA125 using ADCC or CDC. The term “effect” generally refers to a 10% or greater change in ADCC or CDC target cell killing when an agent is incubated with test antibody alone as compared to antibody with CA125. It may, depending on the antibody and the agent used also refer to a change of at least 5%, 15%, 20%, 25%, 30%, 35%, 40, 45%, 50%, 55%, 60%, 70%, or 75%.

Various terms and terminology relating to aspects of the enclosed description are used throughout the specification and claims of this document. Such terms are to be given their ordinary meaning in the art unless otherwise specifically indicated. Other specifically defined terms are to be construed in a manner consistent with the definitions provided.

As used in this specification and the appended claims, the singular forms of “a,” “an,” and “the” also include plural references unless the content clearly specifically dictates otherwise. As example, reference to “a cell” may include a combination of two or more cells, and the like. Reference to “a probe” may include test antibody, an enhancer protein or an independent probe to monitor humoral immune response via any analytical method known by those skilled in the art.

The term “about” as used when referring to quantified values such as an amount, a period of time, and/or the like, is meant to encompass variations of up to ±9% from the specified value, as such variations are appropriate to carry out the disclosed methods. Unless otherwise indicated, all values expressing quantities of reagents, such as molecular weight, molarity, reaction conditions, percentage and so forth used in the specification and claims are to be understood as being quantified in all instances by the term “about.” Accordingly, unless indicated to the contrary, the numerical values as set forth in the following specifications and listed claims are approximations that may vary depending upon the desired properties of the composition agent and/or methods sought to be obtained by the present invention. At the very least, and not as an attempt to limit the scope of the application, each numerical value should at least be valued by the reported significant digits and through ordinary rounding methods known by those skilled in the art.

The term “antibody” as used here is meant in a broad sense and includes immunoglobulin (also referenced as “Ig”) or antibody molecules including polyclonal antibodies (also referenced as pAbs), monoclonal antibodies (also referenced as mAbs) including murine, human, humanized and chimerized mAbs, bispecific antibodies (also referenced as BSP), and antibody fragments. In general, antibodies are proteins or polypeptide chains that bind to a specific antigen. An antigen is a structure that is specifically recognized by a given antibody. Canonical antibodies comprise a hetero-tetramer of glycosylated proteins, composed of two light chains and two heavy chains lined through a complex of disulfide and hydrogen bonds. The term “its disulfide bridge” refers to the disulfide bridge contained within the heavy chain hinge region, which is commonly known by those skilled in the art. Each heavy chain has a variable domain (variable region) (VH) followed by a number of constant domains (referred to as the Fc domain). Each light chain has a variable domain (VL) and a constant domain; the constant domain of the light chain is aligned with the first constant domain of the heavy chain and the light chain VL is aligned with the variable domain of the heavy chain. Antibody light chains of any species are assigned to one of two distinct types based on their amino acid sequences within their constant domains, namely kappa (κ) and lambda (λ).

Immunoglobulins are categorized as classes or isotypes, depending upon the type of Fc domain namely IgA, IgD, IgE, IgG and IgM, which depend on the sequences contained within their heavy chain constant domain. The IgA and IgG isotypes are further comprised of subclasses as the isotypes IgA₁, IgA₂, IgG₁, IgG₂, IgG₃ and IgG₄.

An immunoglobulin VL or VH region consists of a “framework” (FW) region interrupted by three “antigen-binding sites” also referred to as Complementarity Determining Regions (CDRs) based on sequence variability as reported (Wu TT, Kabat EA. J Exp Med 132:211-250, 1970). In general, an antigen-binding site is composed of six CDRs with three located within the heavy chain (CDRH1, CDRH2, CDRH3), and three within the light chain (CDRL1, CDRL2, CDRL3) variable domains (Kabat EA, et al. 5^{th} Ed. PHS, National Institutes of Health, Bethesda, Md., 1991).

“Specific binding” or “specifically binds” refers to the binding of an antibody or antigen-binding fragment to an antigen (including sequences contained within an antibody itself) with greater affinity than for other antigens. Typically, a specific antibody or antigen-binding fragment binds target antigen with an equilibrium dissociation constant K_{D} of about 5x10⁻⁸ M or less.

The term “antibody dynamic structure” refers to any change in structure that can affect antibody humoral function (i.e., Fc receptor or Clq binding, etc.).

The term “monoclonal antibody (mAb)” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology but may also include recombinant methods.

“Fab domain” refers to any antibody sequence N-terminal to the antibody hinge disulfide region which is known by those skilled in the art.

“Fc domain” refers to any antibody sequence C-terminal to and including the antibody hinge disulfide region which is known by those skilled in the art.

The “affected domain” refers to the region located on the rituximab heavy chain (SEQ ID NO: 45) identified within SEQ ID NO: 3.

An “antigen” is an entity to which an antibody or antibody fragment specifically binds. This includes binding to an antibody or protein of interest.

The term “parental antibody” refers to the rituximab antibody composed of SEQ ID NO: 6 and SEQ ID NO: 47.

The term “RTX-MT” refers to an antibody composed of SEQ ID NO: 6 and a heavy chain containing one or more amino acid changes within the affected domain (SEQ ID NO: 3).

The term “CA125-refractory” refers to an RTX-MT antibody that has better ADCC and/or CDC than the parental rituximab antibody, particularly in the presence of CA125.

The term “agent” or “enhancer protein” refers to any protein able to block or reduce CA125 binding to an affected antibody or parental antibody, including the C3B (SEQ ID NO: 1) and the C4B (SEQ ID NO:2) proteins.

The term “affected antibody” refers to an antibody whose humoral immune function is reduced by CA125.

The term “rituximab” refers to the FDA approved antibody [FDA Reference ID: 4274293 (world wide web address: accessdata.fda.gov/drugsatfda _docs/label/2018/103705s5450lbl.pdf)] or any antibody containing the CDR sequences encoding SEQ ID NOs: 39, 40, 41, 42, 43, 44.

The term “CD20” refers to the human cell surface protein expressed by B-cells and is the target antigen to which rituximab specifically binds.

The term “CA125” refers to the gene product produced by MUC16 gene (HGNC: 15582; OMIM: 606154), which is found in soluble and membrane-bound forms. It has been reported to bind to antibodies and affect bound antibody humoral immune function (Kline JB, et al. Oncotarget 8:52045-52060, 2017).

The terms “cancer,” “malignant,” “dysregulated,” and “tumor” are well known in the art and refer to the presence of cells with unregulated cell growth and morphological features different than a normal cell type of similar origin, also referred to as dysregulated cells. Malignant refers to those cancer cells capable of causing morbidity and/or mortality. As used, “cancer and tumor” includes premalignant and malignant types.

As used, the term “soluble” refers to a protein or non-protein agent that is not attached to the cellular membrane of a cell. For example, an agent that is soluble may be shed, secreted or exported from normal or cancerous cells into biological fluids including serum, whole blood, plasma, urine or microfluids of a cell, including tumors.

The “level” of a specified protein or non-protein agent including enhancer proteins or RTX-MTantibodies and CA125, as used, refers to the level or levels of the agent as determined using any method known in the art for the measurement of protein and/or non-protein agent levels in vitro or in vivo. Such methods include gel electrophoresis, capillary electrophoresis, high performance liquid chromatography (HPLC), thin layer chromatography (TLC), hyperdiffusion chromatography, fluid or gel precipitation reactions, absorption spectroscopy, colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, fluorescence resonance energy transfer (FRET), Förster resonance energy transfer, electrochemiluminescence immunoassay, and the like. In one embodiment, the level of CA125 is determined using probe-based techniques, as described in more detail.

The term “humoral immuno-suppression or humoral immune suppression” refers to any antibody, antibody fragment, or bispecific antibody (BSP) that is directly bound by CA125 and whose dynamic structure is altered. It has been reported that CA125 is induced by lymphomas from normal surrounding epithelial cells (Sanusi et al. Perit Dial Int. 21:495-500, 2001) and can bind certain antibodies and alter their dynamic structure thus affecting their biological activities including ADCC, CDC, opsinization, internalization and/or PK, PD and PL profiles.

The term “bispecific antibody (BSP)” refers to any antibody that can bind two or more different antigens. A BSP can comprise at least but not limited to two full length antibodies, a full length antibody and a single chain antibody, or two single chain antibodies, wherein each one binds to different antigens or different epitopes on the same antigen.

The term “antibody dependent cellular cytotoxicity (ADCC)” refers to an in vitro or in vivo process where an antibody can bind to an antigen on a surface of a cell then engage with immune-effector cells via sequences within the antibody’s Fc domain that in turn results their release of toxins that can kill bound cell.

The term “complement dependent cytotoxicity (CDC)” refers to an in vitro or in vivo process where an antibody can bind to an antigen on a surface of a eukaryotic or prokaryotic cell then engage with the C1q protein via sequences within the antibody’s Fc domain that in turn results in initiation of classical complement cascade that can kill bound cell.

The term “opsonization” refers to a process where an antibody can bind to an antigen on a surface of a cell then engage with immune cells via sequences within its Fc domain that in turn results in immune cells engulfing, consuming and ultimately killing antibody bound cell.

The term “pharmacokinetic (PK)” refers to the time that an antibody maintains its steady-state concentration when administered to a subject.

The term “pharmacodynamic (PD)” refers to the study of the biochemical and physiological effects of an antibody-based drug and its mechanisms of action(s), including the correlation of their actions and effects with their biochemical structure when administered to a subject.

The term “pharmacologic (PL)” refers to the known effect an antibody has on managing or killing a disease cell in vitro or in vivo.

The term “sample” refers to a collection of similar fluids, cells or tissues isolated from a subject, as well as fluids, cells or tissues present within a subject. Fluids may include biological fluids that include liquid solutions contacted with a subject or biological source, including cell and organoid culture medium, urine, salivary, lavage fluids and the like.

The term “control sample,” as used, refers to any clinically or non-clinically relevant control sample, including, for example, a sample from a healthy subject not afflicted with a particular cancer type or a cell that is different from its parental cell.

The term “control level” refers to an accepted or pre-determined level of a protein or non-protein agent that is used to compare with the level of the same agent in a sample derived from a subject or used in in vitro assays.

As used, “a difference” between signal of an antibody in control settings vs being bound by CA125 in the presence or absence of an enhancer protein is generally any difference that can be determined using statistical methods commonly used by those skilled in the art and at a minimum a difference of 10% or greater as compared to control. It may, depending on the antibody and the probes used, also refer to a change of at least 5 %, 15%, 20%, 25%, 30%, 35%, 40%, 45%, 50%, 55%, 60%, 70%, or 75%.

The term “inhibit” or “inhibition of” means to reduce by a statistically measurable amount, or to prevent entirely.

The term “functional,” in the context of an antibody, antibody containing moiety (i.e. BSP, etc.) or enhancer protein to be used in accordance with the methods described, indicates that the antibody and/or enhancer protein is capable of reducing or blocking CA125 binding to an affected antibody, respectively, and/or is able to kill target cells in vitro or in vivo better than parental antibody alone.

The term “target cell” refers to a eukaryotic or prokaryotic cell or population of cells that expresses antigen for a specific antibody or antibody containing moiety.

The term “pharmaceutically acceptable” refers to a substance that is acceptable to administer to a patient from a pharmacological as well as toxicological aspect and is manufactured using approaches known by those skilled in the art. These include agents approved by a regulatory agency of the Federal or a state government or listed in the U.S. Pharmacopeia or other generally recognized pharmacopeia for use in animals and humans. The term “pharmaceutically compatible ingredient” refers to a pharmaceutically acceptable diluent, adjuvant, excipient or matrix vehicle with which an anti-cancer agent is administered. “Pharmaceutically acceptable carrier” refers to a matrix that does not interfere with the effectiveness of the biological activity of the active ingredient(s) and is nontoxic to the host.

The terms “effective amount” and “therapeutically effective” are used interchangeably and, in the context of administering a pharmaceutical agent at an amount that is sufficient to produce an enhanced clinical outcome in a patient. An effective amount of an agent is administered according to the methods described here in an “effective regimen.” The term “effective regimen” refers to a combination of amount of the agent and dosage frequency adequate to accomplish an enhanced clinical outcome for a patient with a particular cancer. Enhanced efficacy is an improved clinical outcome when a patient is administered an agent that is capable of overcoming morbidity better than a parental compound or an agent that can enhance the clinical outcome of an effective regimen. As in context here, effective amount refers to the amount of RTX-MT and/or enhancer protein required to demonstrate efficacy or a difference when compared to parental antibody or antibody treatment without an enhancer protein.

The terms “patient” and “subject” are used interchangeably to refer to humans and other non-human animals, including veterinary subjects, that receive a therapeutic agent treatment. The term “non-human animal” includes all vertebrates. In one embodiment, the subject is a human.

“Therapeutic agents” are typically substantially free from undesired contaminants. This means that an agent is typically at least about 50% w/w (weight/weight) pure as well as substantially free from interfering proteins and contaminants.

The term “immune-effector cell” refers to any cell including but not limited to NK, myeloid, monocytes, dendritic cells that may confer antibody dependent cellular cytotoxicity (ADCC) or phagocytosis (opsonization) upon binding to antibody-bound target cell. Cells may be purified or present in mixture in the form of peripheral blood mononuclear cells (PBMCs).

Inflammatory diseases include auto-immune diseases, as well as rheumatoid arthritis, granulomatosis with polyangiitis, idiopathic thrombocytopenic purpura, pemphigus vulgaris, myasthenia gravis and Epstein-Barr virus-positive mucocutaneous ulcers.

The term “dysregulated cell” refers to any cell that is deemed abnormal relative to parental cells. These include transformed cells, malignant cells, virally infected cells, autonomously growing cells via autoregulation, or prokaryotic pathogens.

The term “humoral response” refers to ADCC, CDC or internalization of antibody into target cells by test antibody.

The term “screening” may refer to testing of proteins that can bind to CA125 in the presence of an affected antibody or antibody-containing moiety (i.e., BSP, ADC, single chain antibody, antibody fragment, etc.) and looking for enhanced biological response monitoring ADCC or CDC killing. The term may be used in other contexts in which a large number of test elements is being assayed to determine which among the test elements has a certain property. Similarly, it can be used to refer to the assaying of patient samples for those having a particular property.

The term “significant(ly)” refers to statistical results where the P value as determined by any number of programs including the Student’s T-Test is less than 0.05.

### Composition of Therapeutic Enhancer Proteins and Modified Rituximab Antibodies; and Methods for Developing and Overcoming CA125-Mediated Humoral Immune Suppression

Enhancer proteins (agents) and other chemical or biological agents can effectively block CA125’s suppression of humoral immune response by affected parental and RTX-MT antibodies. In some embodiments, the methods for identifying optimal enhancer proteins and RTX-MT antibodies involve generating RTX-MT antibodies containing one or more amino acid changes in the affected domain (SEQ ID NO: 3) and testing for the ability of RTX-MT antibody to have significantly improved biological activity when used to mediate ADCC or CDC killing against CD20-expressing target cells. In some embodiments, the RTX-MT antibody consists of one amino acid change. In other embodiments, the RTX-MT antibody consists of two or more amino acid changes. Optimal amino acid changes can be determined using functional ADCC or CDC killing assays in the presence of RTX-MT antibody, CA125 and CD20-expressing target cells. Examples are schematically shown in FIG. 1A. Identification of CA125-refractory RTX-MT antibodies that are able to significantly overcome humoral immuno-suppression by CA125 are identified by employing ADCC or CDC killing assays commonly used by those skilled in the art.

In yet another embodiment, the RTX-MT antibody has a binding affinity similar or greater than the parental RTX antibody.

In some methods for identifying CA125-refractory RTX-MT antibodies, antibody is added to a culture of target cells in which target cells naturally or recombinantly express CD20 and CA125. Cultures for comparing humoral responses in the presence of RTX-MT vs parental antibody can be monitored using standard ADCC or CDC killing assays. A change in at least 10% is typically considered as being a meaningful effect on ADCC and/or CDC functions. Depending on the antibody and the assay employed, a meaningful effect also may be defined as a change of at least 5%, 15%, 20%, 25%, 30%, 35%, 40%, 45%, 50%, 55%, 60%, 70%, or 75%. The target cell line may be an engineered cancer cell that naturally expresses CA125 and is transduced with a human CD20 expression construct.

Also provided here are methods of treating a cancer subject with a CA125-refractory RTX-MT antibody. For example, a patient may have a CD20-expressing cancer such as, but not limited to, Hodgkin’s Lymphoma, Non-Hodgkin’s Lymphoma, Follicular Lymphoma, Large Cell Lymphoma, or Chronic Lymphocytic Leukemia. Several reports have found CA125 expression elevated in patients with such cancers and rituximab has been reported to be less effective in patients with CA125 serum levels above the normal range. In such a case, a RTX-MT antibody may be a desirable entity.

In some embodiments of the methods of treating a subject with a RTX-MT antibody, a patient with a CD20⁺ cancer that expresses CA125 may be treated with a RTX-MT antibody alone or in combination with standard-of-care therapy. In some embodiments of the methods of treating a subject with CA125-expressing cancer described here, a RTX-MT antibody is administered to the subject, where the subject has a baseline CA125 level that is above the normal range. In some embodiments of the methods of treating a subject with CA125-expressing cancer described here, the method involves administering the RTX-MT antibody alone. In yet another embodiment, the RTX-MT antibody is administered in combination with chemotherapy. The chemotherapy may be any chemotherapeutic agent considered standard-of-care for a particular cancer indication at the time when the subject is treated. In the methods of treatment described here, CA125 expression level may be determined by any means known in the art and defined as within or above the normal range by those skilled in the art.

In some embodiments of the methods of treatment described here, the RTX-MT antibody consists of one amino acid change in the affected domain (SEQ ID NO: 3). The RTX-MT antibody is administered to patients with CA125-expressing, CD20-positive (CD20⁺) cancers. Exemplary CD20-positive cancers known to produce CA125 are Hodgkin’s and Non-Hodgkin’s Lymphoma, Follicular Lymphoma, Large Cell Lymphoma, and Chronic Lymphocytic Leukemia. Other cancers may also be amenable to such treatment, including, without limitation, mesothelioma, ovarian, breast, lung, colorectal, gastro-intestinal, endometrial, and pancreatic cancers.

The present methods can be combined with other means of treatment such as surgery (e.g., debulking surgery), radiation, targeted therapy, chemotherapy, immunotherapy, use of growth factor inhibitors, or anti-angiogenesis factors. A RTX-MT antibody or parent antibody plus enhancer agent can be administered concurrently to a patient undergoing surgery, chemotherapy or radiation therapy treatments. Alternatively, a patient can undergo surgery, chemotherapy or radiation therapy prior to or subsequent to administration of the RTX-MT antibody or parent antibody plus enhancer agent by at least an hour and up to several months, for example at least an hour, five hours, 12 hours, a day, a week, a month, or three months, prior or subsequent to administration of standard of care therapy. Some embodiments of the methods of treatment provided here involve administration of a therapeutically effective amount of chemotherapy plus an affected antibody such as an anti-CD20 antibody to a tumor-specific antigen to the subject in addition to the enhancer protein.

In some embodiments of the methods of treatment described here, the subject may have received first-line surgical resection of the tumor, first-line chemotherapy for treatment of the cancer prior to administering an affected antibody specific to an antigen expressed by said cancer and enhancer protein.

In some embodiments of the methods of treatment described here, the subject may have received first-line surgical resection of the tumor, first-line chemotherapy for treatment of the cancer prior to administering a refractory antibody or parental antibody plus enhancer protein in which the antibody is specific to an antigen expressed by said cancer and the enhancer protein can specifically block a humoral immuno-suppressive protein such as CA125.

Administration of the RTX-MT antibody and/or enhancer proteins in accordance with the methods of treatment described here may be by any means known in the art. Typically, the antibody is infused intravenously.

In yet another embodiment, a therapeutic antibody may be used that comprises the CDR sequences that can direct binding of an antibody to the CD20 antigen: SEQ ID NO: 41 (GYTFTSYN) as CDRH1, SEQ ID NO: 42 (IYPGNGDT) as CDRH2, SEQ ID NO: 43 (ARSTYYGGDWYFNV) as CDRH3, SEQ ID NO: 44 (SSSVSY) as CDRL1, SEQ ID NO: 45 (ATS) as CDRL2 and SEQ ID NO: 46 (QQWTSNPPT) as CDRL3, numbered according to IMGT® (the international ImMunoGeneTics Information System®) and have a mutation with the affected domain (SEQ ID NO: 3). The antibody may be administered to CD20-positive disease indication and where CA125 is above the normal range in a coadministration with an enhancer protein or alone. Dose level of enhancer protein may be as high as the clinically determined maximal tolerated dose (MTD) or levels below the MTD. Administration of an enhancer protein can be prior to, concomitant with or after administration of antibody. Treatment can include surgery as well as treatment with standard-of-care. The enhancer protein or other chemical or biological enhancing agent may be administered along with, in combination with, or separately from the antibody. It may, for example, be administered orally, intradermally, subcutaneously, intramuscularly, or intravenously.

Various delivery systems can be used to administer the RTX-MT antibody or enhancer protein including intradermal, intramuscular, intraperitoneal, intravenous, subcutaneous, intranasal, epidural, and oral routes as deemed necessary. They can be administered, for example by infusion or bolus injection, by absorption through epithelial or mucocutaneous linings (e.g., oral mucosa, rectal and intestinal mucosa, and the like) via systemic or local approaches.

The RTX-MT antibody or enhancer protein can be administered by injection via syringe, catheter, suppository, or any implantable matrix or device.

The RTX-MT antibody or enhancer protein and pharmaceutical compositions thereof for use as described here may be administered orally in any acceptable dosage form such as capsules, tablets, aqueous suspensions, solutions or the like.

Suitable methods of administration of the RTX-MT antibody or enhancer protein include but are not limited to intravenous injection and intraperitoneal administration at a final concentration suitable for effective therapy.

The RTX-MT antibody or enhancer protein in combination with other drugs can be administered as pharmaceutical compositions comprising a therapeutically or prophylactically effective amount of the therapeutic agent(s) and one or more pharmaceutically acceptable or compatible ingredients.

The amount of the therapeutic agent that is effective in the treatment or prophylaxis of a cancer or non-oncologic disease can be determined by standard clinical techniques. In addition, in vitro assays may optionally be employed to help identify optimal dosage ranges required for the RTX-MT antibody or enhancer protein. Effective doses may be extrapolated from dose-response curves of RTX-MT antibody or enhancer protein derived from in vitro or animal model test systems.

For example, toxicity and therapeutic efficacy of the RTX-MT antibody or enhancer protein can be determined in cell cultures or experimental animals by standard pharmaceutical procedures for determining the LD₅₀ (the dose lethal to 50% of the population) and the ED₅₀ (the dose therapeutically effective in 50% of the population) values. The dose ratio between toxic and therapeutic effects is the therapeutic index and it can be expressed as the ratio LD₅₀/ED₅₀. Agents that exhibit large therapeutic indices are suitable. When an agent exhibits toxic side effects, a delivery system that targets the agent to the site of affected tissue can be used to minimize potential damage to non-CD20-expressing cells and, thereby, reduce side effects.

Nucleic acid vectors may be plasmids, viruses, or subviral, for example. Preferably the nucleic acid will be maintained in a desired host cell by having an effective origin of replication, but in some situations, transient expression may be desired. Typically, it will be desirable to have the mutant antibodies expressed, requiring expression control sequences on the vector and appropriate accessory proteins in the host cell. Often, to produce large quantities of an antibody or antibody portion, stable cell lines will be formed that express all or a portion of the antibody. Antibody portions may be entire light or heavy chains, for example.

The dosing and dosage schedule may vary depending on the active drug concentration, which may depend on the needs, size, and status of the subject.

### Example 1-Screening for Proteins that can Overcome CA125 Immuno-Suppression of Affected Antibodies and Mapping of CA125 Antibody Binding

FIGS. 1A-1D shows a screening method and results to identify proteins that can block the CA125 immuno-suppressive effects of affected antibodies and the use of their binding to map regions on affected antibody to which CA125 can bind.

An enhancer protein blocks the immuno-suppressive effect of CA125 on an affected antibody; the affected antibody is rituximab. The screening assay involves a 96-well plate ELISA, whereby wells are coated with rituximab with or without CA125 protein, then wells are probed with a biotinylated human CD16a with or without the C3B or C4B protein and measured for CD16a binding. Previous studies have shown the CA125 can suppress the effects of CD16a or C1q protein binding to affected antibody. As shown in FIG. 1B, wells containing RTX bound both CD16a and C1q proteins while those with RTX plus CA125 had significantly reduced binding of either protein. Next, the C4B protein (SEQ ID NO: 2) was added to wells containing RTX plus CA125. The addition of C4B to the wells containing rituximab plus CA125 resulted in restored CD16a and C1q binding. To confirm this effect resulted in a functional outcome, human CD20-positive Daudi cells were treated with RTX with or without exogenously added CA125 and screened for ADCC or CDC activity as described below. As shown in FIG. 1C, CA125 suppressed CDC activity. Next, C4B was added to similar cultures and found that it could restore CDC activity in wells treated with RTX plus CA125. This effect appears to be a result of C4B binding to RTX antibody and not CA125 as direct binding assays via ELISA find that a biotinylated C4B can bind to wells coated with RTX but not CA125 (not shown). Similar effects were observed for C3B and a subset of data are provided in FIG. 1D.

CD16A activation assays were conducted using Jurkat-Luc effector cells, which are part of the ADCC Reporter Bioassay Core Kit and assayed as recommended by the vendor (Promega). Briefly, 2x10⁴ target cells were seeded overnight in black opaque 96 well plates in triplicate in assay buffer (RPMI+L-glutamine+1% ultra-low Ig serum) (Gibco). The following day, 1x10⁵ Jurkat-Luc effector cells were added to wells in assay buffer for an effector target ratio of 5:1. Antibody, effector and target cells were incubated for 16 hours at 37° C./5%CO₂. Plate was equilibrated at room temperature for 10 minutes. Fifty microliters of Bio-glo reagent (Promega) was added to wells and luminescence quantitated using a Varioskan LUX plate reader (ThermoFisher).

The competition of RTX binding to CD20 in the presence of CA125 was carried out as follows. Briefly, ELISA plates were coated overnight at 4° C. with diluted antigen in 0.05 M carbonate buffer, pH 9.5. Coated antigen was CD20/MS4A1 linear peptide (Rp1-L; Favoino et al., Int J Mol Sci. 2019 Apr; 20(8): 1920) at 10 µg/mL. Plates were blocked with 0.05 M phosphate buffer, pH7.2 (PBS) containing 5% BSA for 1 hr at room temperature then washed twice with PBS. Biotinylated RTX (by EZ-link Sulfo-NHS-LC-Biotin, ThermoFisher) or negative control human serum albumin (HSA) were added to the plate in PBS at 5 µg/mL for 1 hr at room temperature. Competition was carried out by adding in some reactions 10 µg/mL unlabeled RTX (2-fold excess) or 30 KU/mL of CA125. Wells were washed 3 times with PBS followed by addition of streptavidin-HRP in PBS/BSA for 1 hr at room temperature. After washing, reactions were developed for up to 10 minutes by adding 75 µL of TMB substrate (Pierce) and stopped by adding 75 µL 0.1 N H₂SO₄. Absorbance at 450 nm was measured in a Varioskan plate reader (ThermoFisher).

Previous reports have shown that CA125 can bind to the Fab domain of affected antibodies (Kline JB, et al. OncoTarget 8:52045-52060, 2017; Nicolaides NC, et al. Cancer Biol Ther 13:1-22, 2018). C3B and C4B have been previously reported to bind to the Fab domain of IgG1-type antibodies (Campbell RD et al. Biochem J 189:67-80, 1980; Sahu A and Pangburn MK J Biol Chem 269:28997-29002, 1994).

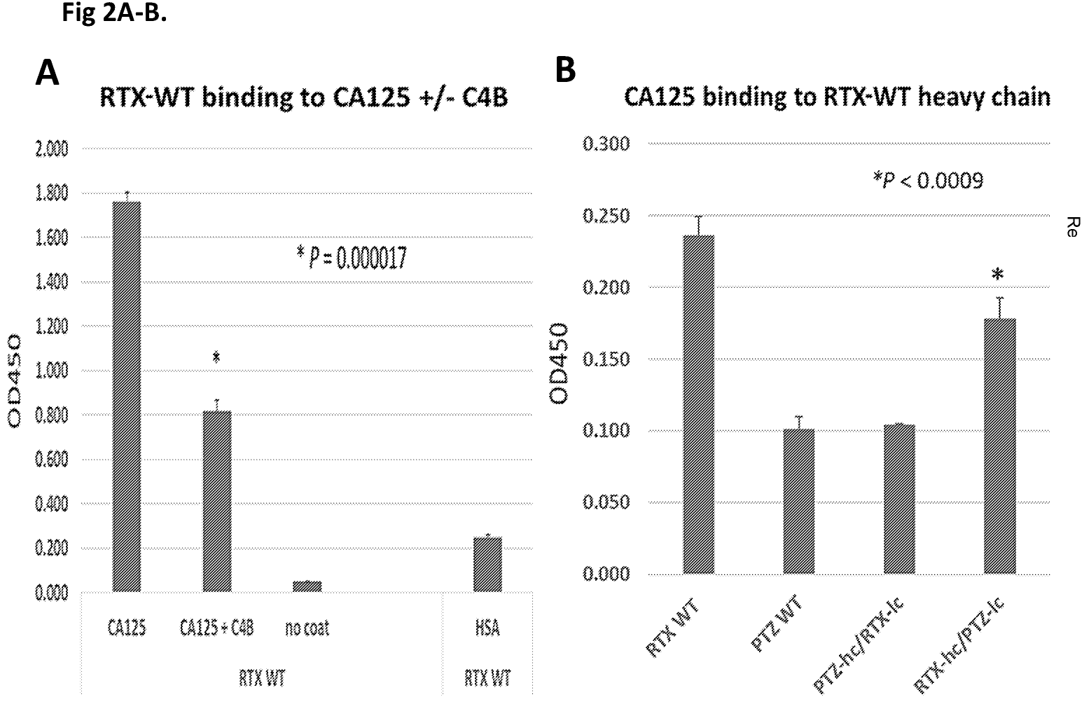

To determine if C3B and/or C4B can block CA125 immuno-suppression via blocking its ability to bind rituximab, competitive binding assays using ELISA were conducted. As shown in FIG. 2A, a biotinylated rituximab was able to bind CA125 as previously reported (Kline JB, et al. OncoTarget 8:52045-52060, 2017). The addition of a recombinant C4B protein was able to block rituximab’s binding to CA125, thereby confirming that the reduced CA125 immuno-suppressive effect was through inhibition of CA125 binding to affected antibody. A similar effect was observed with C3B (FIG. 1D). In contrast other IgG binding proteins such as protein L did not block CA125 binding demonstrating the specificity of thC3B and C4B effect (FIG. 1D.) While the C3B or C4B binding to Fab domain of antibody has been reported, the determination of the immunoglobulin domain to which it binds (light or heavy chain, or both) was equivocal. To define better if CA125 binds to the light, heavy or both immunoglobulin chains of rituximab, chimeric antibodies using the light and heavy chain of the unaffected antibody pertuzumab (PTZ) (Kline JB, et al. OncoTarget 8:52045-52060, 2017) was combined with the heavy and light chain of rituximab to create chimeric antibodies. These antibodies were then biotinylated and tested for CA125 binding. As shown in FIG. 2B, the chimeric antibody containing the rituximab heavy chain (RTX-hc/PTZ-lc) was still able to bind CA125 while the chimeric containing the light chain (PTZ-hc/RTX-lc) was not, thereby refining the putative binding of CA125 to the heavy chain within the region containing the CDR3 and residues N-terminal to the disulfide hinge region. In addition, the rituximab-CA125 interaction could be competed by the complement protein C3B (FIG. 1D), which binds to the CH1 domain of the heavy chain, but not by Protein L, which binds to the light chain variable domain. These data provide evidence that the CA125 binding site is located within the rituximab heavy chain and that C3B (SEQ ID NO: 1) can be used to reverse CA125 immunosuppressive effects.

### Example 2—Identifying Critical Residues Required for CA125 Binding and Generation of CA125-Refractory Rituximab Antibodies

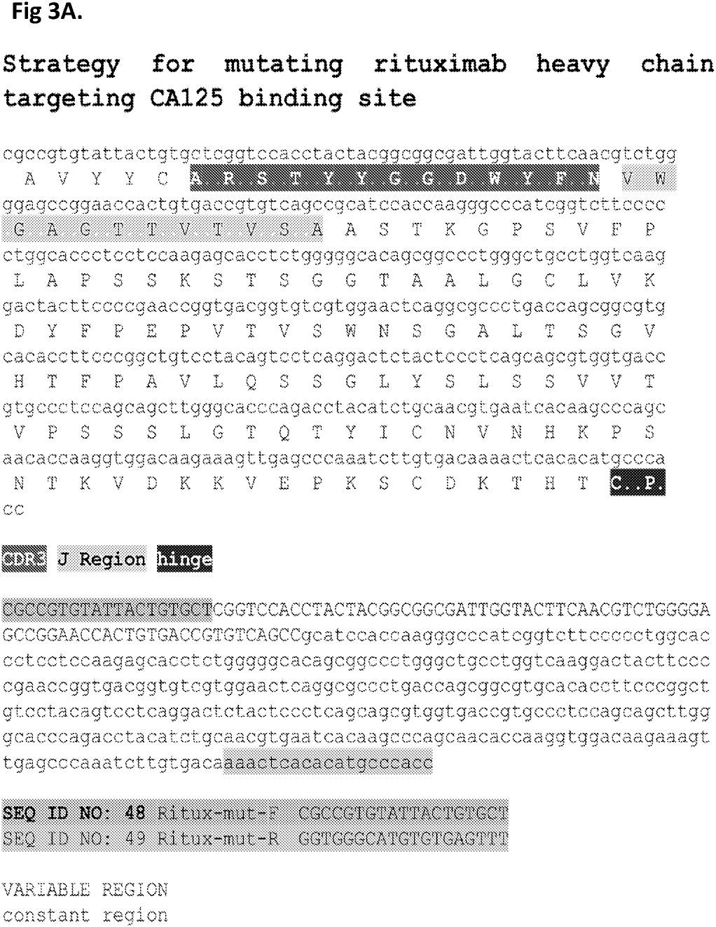

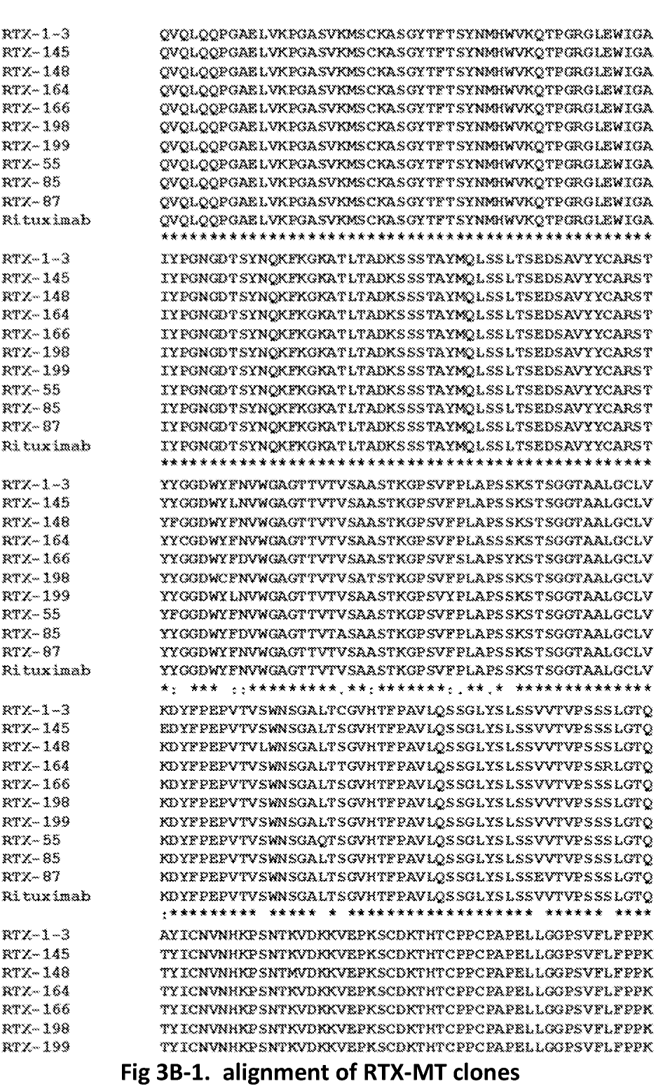

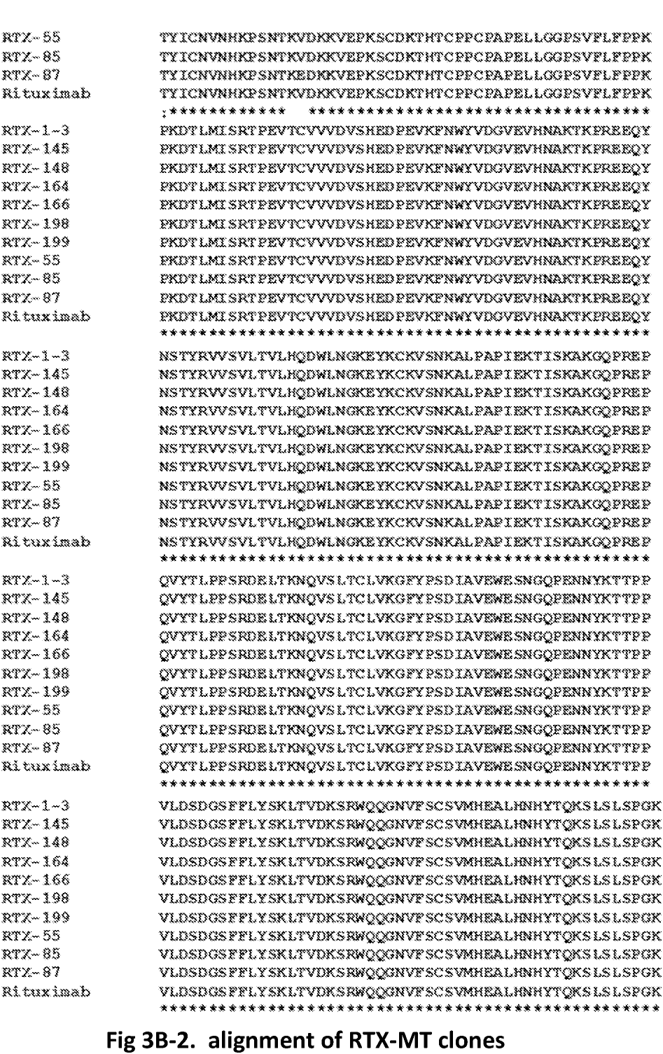

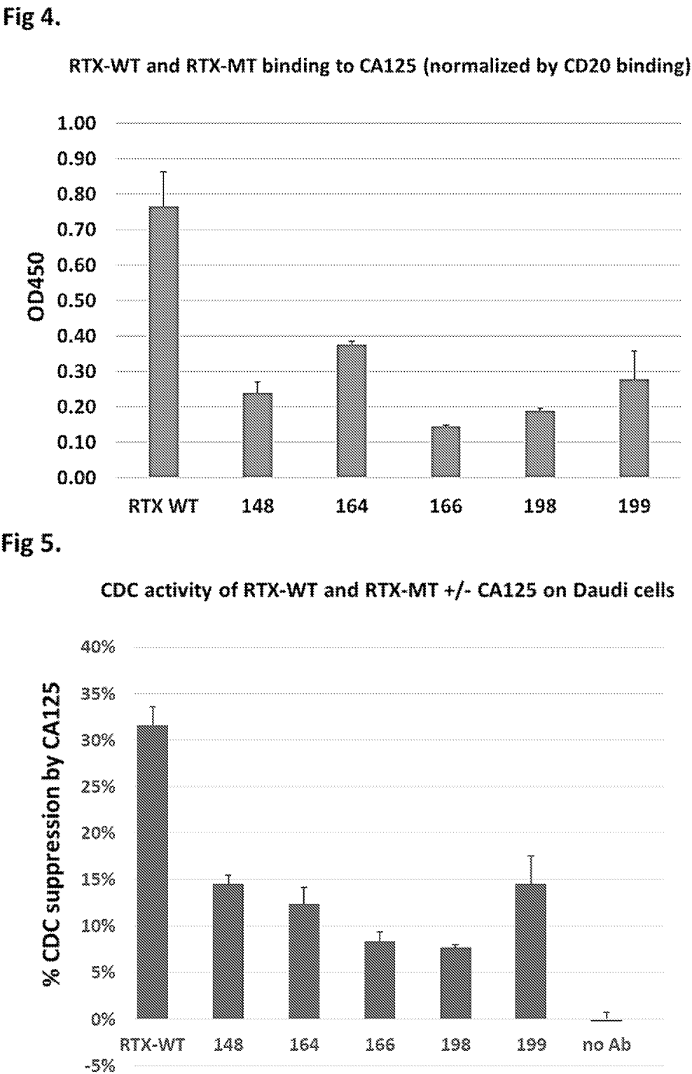

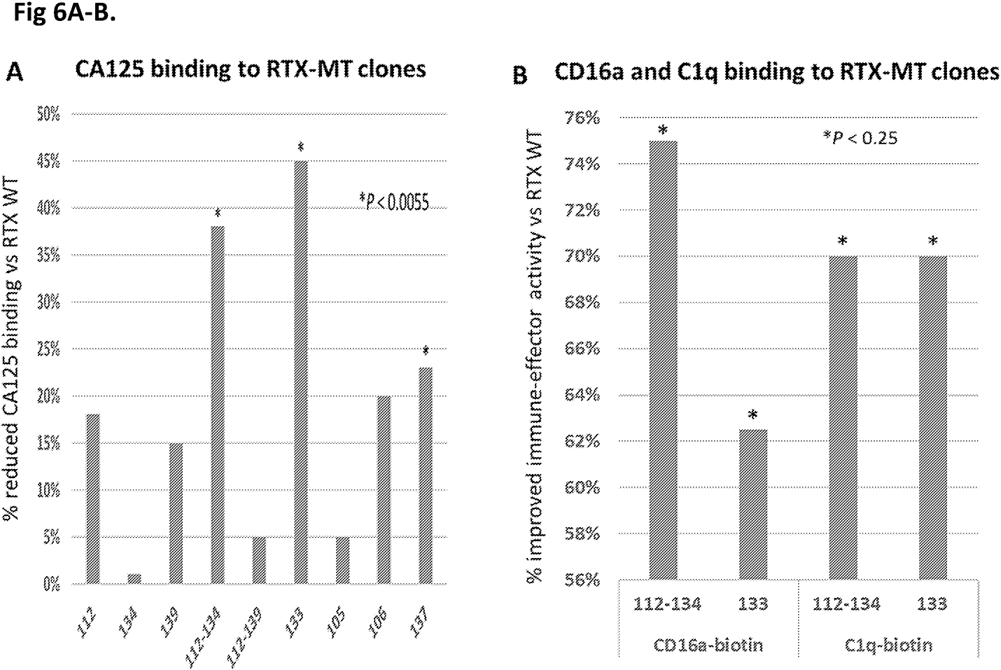

The C3B and C4B proteins have been shown to bind to the CDR3-framework 4 (CDR3-FW4) and CH1 regions of antibodies, respectively, and the results shown in FIGS. 1 and 2 indicate that these proteins compete CA125 binding to parental rituximab (RTX-WT) suggesting that CA125 may bind to CDR3-FW4 and CH1 regions. Because RTX-WT heavy chain is also sufficient for CA125 binding, we applied a random mutagenesis library approach to try to determine if a specific residue or residues are required for CA125 binding to rituximab heavy chain. We employed a random mutagenesis PCR method commonly used by those skilled in the art to create a library of rituximab heavy chains with mutated residues within the affected domains CDR3-FWR4 and CH1 (SEQ ID NO: 3) and then expressed them recombinantly in combination with the rituximab light chain (SEQ ID NO: 6) in HEK293 cells using eukaryotic expression vectors. A library of over 1000 clones were generated. As mutations within the CDR3 may negatively affect CD20 antigen binding, culture supernatants were screened for antibody production and CD20 binding via ELISA. Over 200 rituximab clones were then analyzed for CA125 binding and five were found to retain CD20 binding similar to or greater than the parental antibody and have significantly reduced CA125 binding. The heavy chains from these clones were then sequenced and all were found to have mutations within the targeted region. All clones had multiple mutations and some had overlapping mutated amino acids as shown in FIG. 3. As shown in FIG. 4, clones 148 (SEQ ID NO: 37 and 38), 164 (SEQ ID NO: 31 and 32), 166 (SEQ ID NO: 4 and 5), 198 (SEQ ID NO: 19 and 20) and 199 (SEQ ID NO: 25 and 26) had the most robust CD20 binding and reduced CA125 binding. We expanded these clones, purified their specific RTX-MT antibody and tested them in functional ADCC and CDC assays. As shown in FIG. 5, clones 164 (SEQ ID NOS: 31 and 32), 166 (SEQ ID NOS: 4 and 5) and 198 (SEQ ID NOS: 19 and 20) were the most refractory to CA125. These RTX-MT antibodies are now ready for pre-clinical and clinical testing. To further delineate if one or more mutations within these antibodies were required for reduced CA125 binding, we engineered single or double mutations into the rituximab heavy chain and rescreened for CA125 binding. As shown in FIG. 6A, RTX-MT clone 112-134 (amino acid changes of N to D at codon 109 and P to S at codon 131, SEQ ID NOS: 13 and 14), clone 133 (amino acid change of F to Y at codon 130, SEQ ID NOS: 29 and 30), clone 106 (amino acid change of G to C at codon 103, SEQ ID NOS: 33 and 34), and clone 137 (amino acid change of P to S at codon 134, SEQ ID NOS: 35 and 36) showed reduced binding to CA125 (P < 0.0055) while still retaining CD20 binding like their parental antibody. RTX-MT clones 112-134 and 133 demonstrated improved CD16a-biotin and C1q-biotin binding in the presence of CA125 as compared to the parental antibody (FIG. , 6B), corroborating the link between CA125 or lack thereof binding to antibody and antibody immune-effector complex formation and function.

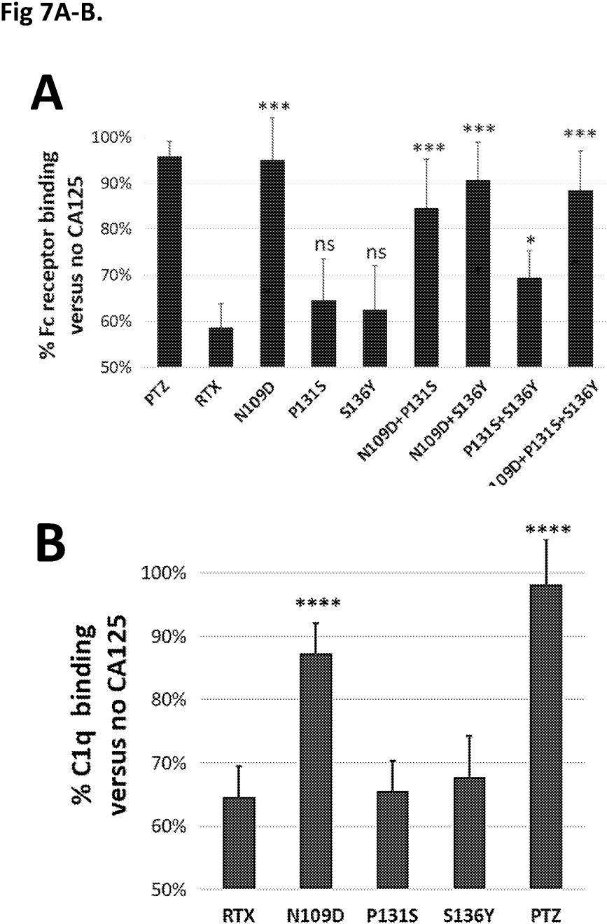

Mutations derived from RTX-MT clone 166 were further studied as it was not obvious whether all the amino acid changes or combinations thereof were required for the observed CA125-refractory phenotype. Sequence analyses showed that the RTX-MT clone 166 had 3 amino acid changes (N to D at codon 109, P to S at codon 131, and S to Y at codon 136 (SEQ ID NO: 5)). In order to study the contribution of each mutation to the observed phenotype, additional RTX-MT variants were engineered each having one of the three amino acid changes found in the RTX-MT clone 166 as well as combinations of double mutations. The analyses of these new RTX-MT variants (named N109D, P131S, S136Y, N109D+P131S, N109D+S136Y and P131S+S136Y) suggested that the presence of the N109D mutation (amino acid change of N to D at codon 109) correlated with reduced sensitivity to CA125-mediated suppression of Fc receptor (FIG. 7A) as well as C1q binding (FIG. 7B). The anti-HER2 antibody pertuzumab (PTZ) was used as a CA125-refractory control antibody. Upon further testing, the N109D variant (hereon referred to as RTX-MT or RTX N109D) was shown to bind to CD20 at levels comparable to parent RTX (FIG. 7C) and, importantly, its binding to CA125 was significantly reduced by 50% (FIG. 7D). Overall, these results indicate that the mutation N109D (change N to D at codon 109) is sufficient to reduce RTX N109D sensitivity to CA125.

RTX N109D was used as an example of CA125-refractory RTX-MT variant and further characterized. Its binding to CD20 was analyzed by an alternative assay to ensure comparability to parent RTX’s CD20 binding. The measurement of antibody equilibrium dissociation constant (Kd) was carried out by using a cell-based fluorescent assay involving the immunostaining of antigen-positive (Ramos) and antigen-negative (Jurkat) cell lines. In this assay, cells are stained with a 2-fold concentration titration of the 488-labeled antibody ranging from 0.78 to 100 nM. Briefly, cells are grown in suspension in complete RPMI. Confluent cultures with viability >85% are centrifuged at 800 RPM for 6 min and cell pellets are resuspended in 4 mL of Animal-Free Blocker (Vector Laboratories SP-5035) in a 50-mL tube to wash the cells. Cells are centrifuged again and resuspended in Animal-Free Blocker at 10 million/mL. Samples are incubated for 20 min on ice to block any non-specific binding sites. Cells (200 mL/2 million cells/well) are then transferred in V-bottom, polypropylene 96-well microplate (Corning 3363) and centrifuged in a benchtop centrifuge at 1,500 RPM for 2 min. Supernatants are removed and the cells are resuspended in 90 mL/well of Animal-Free Blocker. Each condition is tested in duplicate wells. Diluted 488-labeled antibody (10 µL/well) is added to the corresponding well and samples are incubated for 1 hour on ice with mixing every 20 min. Cells are then washed 4 times using 100 µL/well of Animal-Free Blocker and centrifuged at 1,500 RPM for 2 min after each wash. After the last wash, cells are fixed with 100 µL/sample of 10% Formalin (LabChem, LC146702) for 10 minutes. Samples are centrifuged again and then cells are resuspended in 100 µL/well of Animal-Free Blocker and transferred into a black microplate (Greiner Bio-One, 655086). The fluorescence at 494 nm excitation/519 nm emission for each sample were measured by using a Varioskan LUX plate reader. The non-specific background binding to antigen-negative cells is subtracted from the binding signal from the antigen-positive cells. Subtracted values are plotted using a nonlinear regression analysis for “saturation binding with one site” in GraphPad Prism version 8.4. The model determines the Kd as being the ligand concentration that binds to half the receptor sites at equilibrium.

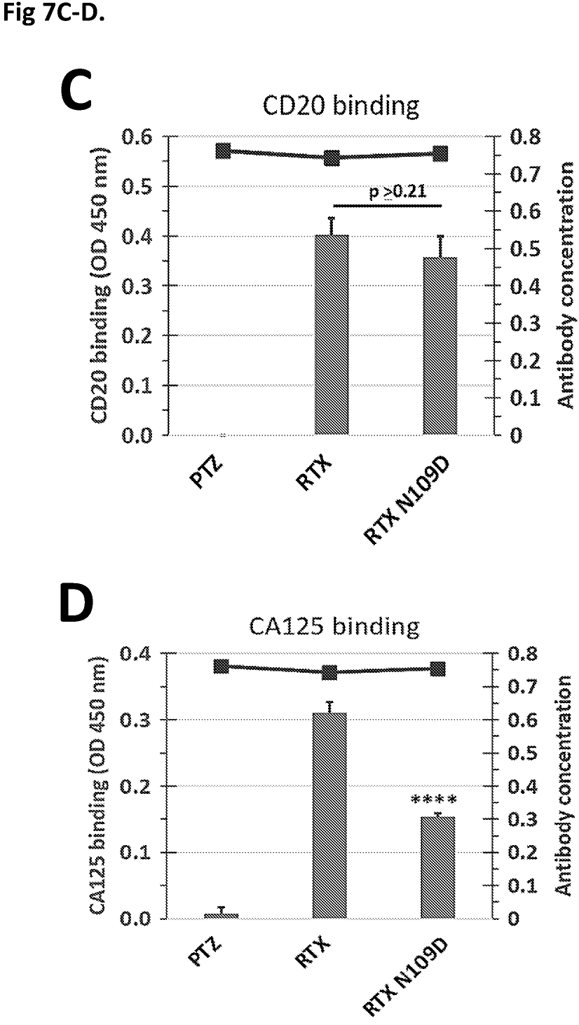

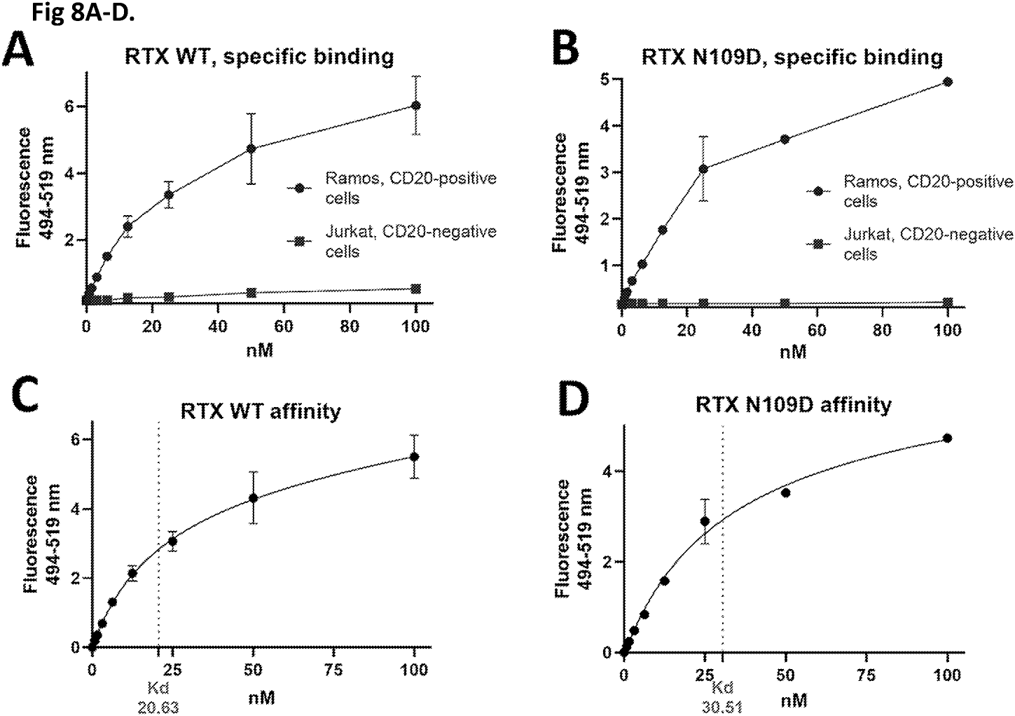

Using this binding assay, RTX N109D bound to CD20-positive Ramos cells in a dose-dependent fashion, while it did not bind to CD20-negative Jurkat cells confirming retention of target cells specificity similar to parent rituximab (RTX) (FIGS. 8A-8B). Moreover, the measured affinity/Kd for RTX and RTX N109D were 20.6 and 30.5 nM, respectively (FIGS. 8C-8D). While a small drop in RTX N109D affinity was expected and consistent with previous CD20 binding data using a different assay (FIG. 7C), overall RTX N109D showed CD20-specific binding and affinity comparable to parental rituximab.

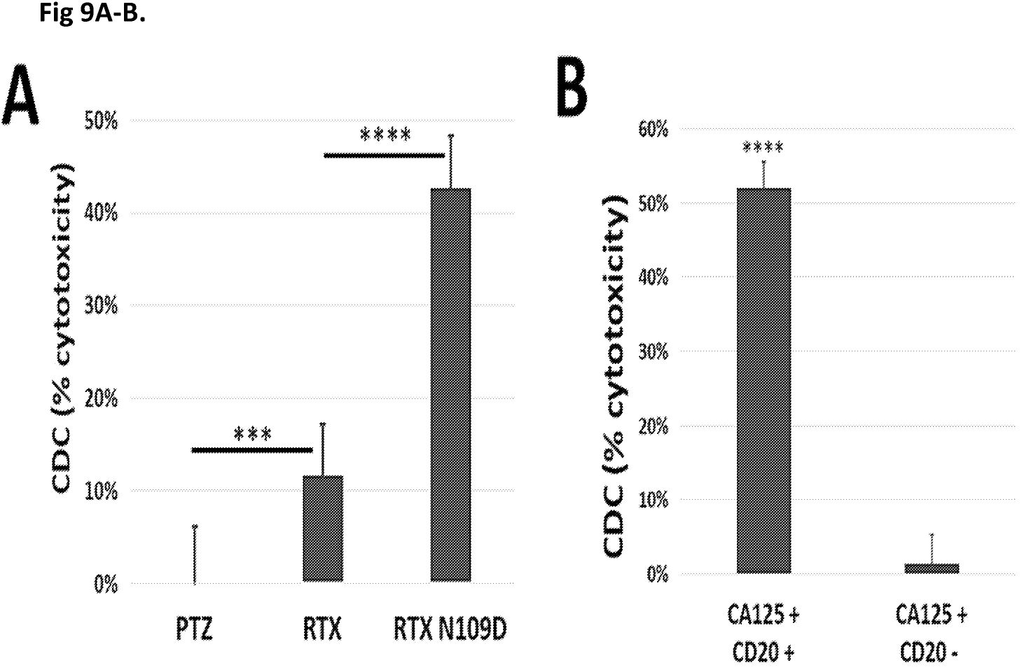

Since CA125 inhibits C1q binding to parental rituximab, leading to reduced CDC activity, the effects of CA125 on CDC mediated by RTX N109D was investigated. As shown previously, C1q binding to RTX N109D was increased compared to parental rituximab (RTX) (FIG. 7B). This increase in C1q binding resulted in enhanced CDC activity mediated by RTX N109D compared to RTX using target cells expressing immunosuppressive CA125 as well as CD20 (FIG. 9A), while no significant killing was noted when using CA125-positive but CD20-negative cells (FIG. 9B).

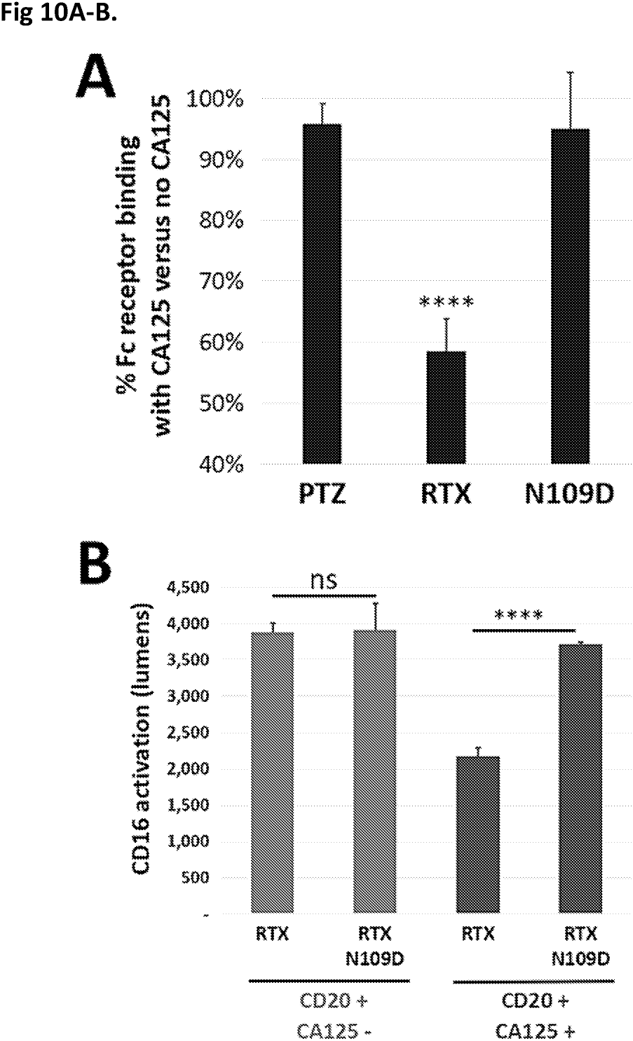

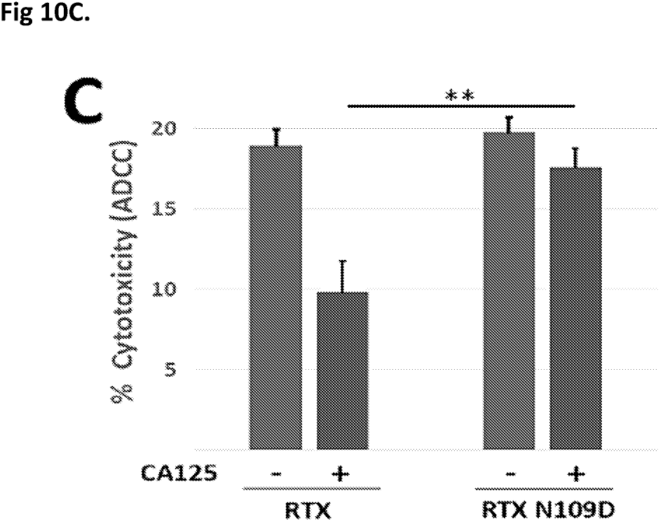

Because CA125 inhibited parental rituximab binding to Fc receptor as well as its signaling activation and subsequent reduced ADCC activity, we investigated whether this immunosuppressive effect was lessened when using RTX N109D. The analysis showed that RTX N109D bound to Fc receptor as well as activated its signaling at significantly higher levels than parent rituximab (RTX) in target cells that expressed CA125, while no significant difference was noted when using CA125-negative cells (FIGS. 10A-10B). PTZ was used as a CA125-refractory control antibody. An important consequence of the enhanced Fc receptor interaction and activation was that RTX N109D was able to mediate a more robust Fc receptor-dependent cytotoxic activity (ADCC) against target cells in the presence of the immunosuppressive CA125 than the parental rituximab (RTX) (FIG. 10C).

These data demonstrate that modifying residues within the affected region (SEQ ID NO: 3) of rituximab heavy chain to generate CA125-refractory antibodies may improve CD20-positive cell killing in the presence of the CA125 immunosuppressive protein.

All references cited in this document are expressly incorporated herein.

### Sequence Identification (All Sequences N to C, for RTX-MT Amino Acid Sequences, Amino Acid Change Indicated in Bold

SEQ ID NO: 1 C3B

MGPTSGPSLLLLLLTHLPLALGSPMYSIITPNILRLESEETMVLEAHDAQ
GDVPVTVTVHDFPGKKLVLSSEKTVLTPATNHMGNVTFTIPANREFKSEK
GRNKFVTVQATFGTQVVEKVVLVSLQSGYLFIQTDKTIYTPGSTVLYRIF
TVNHKLLPVGRTVMVNIENPEGIPVKQDSLSSQNQLGVLPLSWDIPELVN
MGQWKIRAYYENSPQQVFSTEFEVKEYVLPSFEVIVEPTEKFYYIYNEKG
LEVTITARFLYGKKVEGTAFVIFGIQDGEQRISLPESLKRIPIEDGSGEV
VLSRKVLLDGVQNPRAEDLVGKSLYVSATVILHSGSDMVQAERSGIPIVT
SPYQIHFTKTPKYFKPGMPFDLMVFVTNPDGSPAYRVPVAVQGEDTVQSL
TQGDGVAKLSINTHPSQKPLSITVRTKKQELSEAEQATRTMQALPYSTVG
NSNNYLHLSVLRTELRPGETLNVNFLLRMDRAHEAKIRYYTYLIMNKGRL
LKAGRQVREPGQDLVVLPLSITTDFIPSFRLVAYYTLIGASGQREVVADS
VWVDVKDSCVGSLVVKSGQSEDRQPVPGQQMTLKIEGDHGARWLVAVDKG
VFVLNKKNKLTQSKIWDVVEKADIGCTPGSGKDYAGVFSDAGLTFTSSSG
QQTAQRAELQCPQPAARRRRSVQLTEKRMDKVGKYPKELRKCCEDGMREN
PMRFSCQRRTRFISLGEACKKVFLDCCNYITELRRQHARASHLGLARSNL
DEDIIAEENIVSRSEFPESWLWNVEDLKEPPKNGISTKLMNIFLKDSITT
WEILAVSMSDKKGICVADPFEVTVMQDFFIDLRLPYSVVRNEQVEIRAVL
YNYRQNQELKVRVELLHNPAFCSLATTKRRHQQTVTIPPKSSLSVPYVIV
PLKTGLQEVEVKAAVYHHFISDGVRKSLKVVPEGIRMNKTVAVRTLDPER
LGREGVQKEDIPPADLSDQVPDTESETRILLQGTPVAQMTEDAVDAERLK
HLIVTPSGCGEQNMIGMTPTVIAVHYLDETEQWEKFGLEKRQGALELIKK
GYTQQLAFRQPSSAFAAFVKRAPSTWLTAYVVKVFSLAVNLIAIDSQVLC
GAVKWLILEKQKPDGVFQEDAPVIHQEMIGGLRNNNEKDMALTAFVLISL
QEAKDICEEQVNSLPGSITKAGDFLEANYMNLQRSYTVAIAGYALAQMGR
LKGPLLNKFLTTAKDKNRWEDPGKQLYNVEATSYALLALLQLKDFDFVPP
VVRWLNEQRYYGGGYGSTQATFMVFQALAQYQKDAPDHQELNLDVSLQLP
SRSSKITHRIHWESASLLRSEETKENEGFTVTAEGKGQGTLSVVTMYHAK
AKDQLTCNKFDLKVTIKPAPETEKRPQDAKNTMILEICTRYRGDQDATMS
ILDISMMTGFAPDTDDLKQLANGVDRYISKYELDKAFSDRNTLIIYLDKV
SHSEDDCLAFKVHQYFNVELIQPGAVKVYAYYNLEESCTRFYHPEKEDGK
LNKLCRDELCRCAEENCFIQKSDDKVTLEERLDKACEPGVDYVYKTRLVK
VQLSNDFDEYIMAIEQTIKSGSDEVQVGQQRTFISPIKCREALKLEEKKH
YLMWGLSSDFWGEKPNLSYIIGKDTWVEHWPEEDECQDEENQKQCQDLGA
FTESMVVFGCPN

SEQ ID NO: 2 C4B

MRLLWGLIWASSFFTLSLQKPRLLLFSPSVVHLGVPLSVGVQLQDVPRGQ
VVKGSVFLRNPSRNNVPCSPKVDFTLSSERDFALLSLQVPLKDAKSCGLH
QLLRGPEVQLVAHSPWLKDSLSRTTNIQGINLLFSSRRGHLFLQTDQPIY
NPGQRVRYRVFALDQKMRPSTDTITVMVENSHGLRVRKKEVYMPSSIFQD
DFVIPDISEPGTWKISARFSDGLESNSSTQFEVKKYVLPNFEVKITPGKP
YILTVPGHLDEMQLDIQARYIYGKPVQGVAYVRFGLLDEDGKKTFFRGLE
SQTKLVNGQSHISLSKAEFQDALEKLNMGITDLQGLRLYVAAAIIESPGG
EMEEAELTSWYFVSSPFSLDLSKTKRHLVPGAPFLLQALVREMSGSPASG
IPVKVSATVSSPGSVPEVQDIQQNTDGSGQVSIPIIIPQTISELQLSVSA
GSPHPAIARLTVAAPPSGGPGFLSIERPDSRPPRVGDTLNLNLRAVGSGA
TFSHYYYMILSRGQIVFMNREPKRTLTSVSVFVDHHLAPSFYFVAFYYHG
DHPVANSLRVDVQAGACEGKLELSVDGAKQYRNGESVKLHLETDSLALVA
LGALDTALYAAGSKSHKPLNMGKVFEAMNSYDLGCGPGGGDSALQVFQAA
GLAFSDGDQWTLSRKRLSCPKEKTTRKKRNVNFQKAINEKLGQYASPTAK
RCCQDGVTRLPMMRSCEQRAARVQQPDCREPFLSCCQFAESLRKKSRDKG
QAGLQRALEILQEEDLIDEDDIPVRSFFPENWLWRVETVDRFQILTLWLP
DSLTTWEIHGLSLSKTKGLCVATPVQLRVFREFHLHLRLPMSVRRFEQLE
LRPVLYNYLDKNLTVSVHVSPVEGLCLAGGGGLAQQVLVPAGSARPVAFS
VVPTAATAVSLKVVARGSFEFPVGDAVSKVLQIEKEGAIHREELVYELNP
LDHRGRTLEIPGNSDPNMIPDGDFNSYVRVTASDPLDTLGSEGALSPGGV
ASLLRLPRGCGEQTMIYLAPTLAASRYLDKTEQWSTLPPETKDHAVDLIQ
KGYMRIQQFRKADGSYAAWLSRGSSTWLTAFVLKVLSLAQEQVGGSPEKL
QETSNWLLSQQQADGSFQDLSPVIHRSMQGGLVGNDETVALTAFVTIALH
HGLAVFQDEGAEPLKQRVEASISKASSFLGEKASAGLLGAHAAAITAYAL
TLTKAPADLRGVAHNNLMAMAQETGDNLYWGSVTGSQSNAVSPTPAPRNP
SDPMPQAPALWIETTAYALLHLLLHEGKAEMADQAAAWLTRQGSFQGGFR
STQDTVIALDALSAYWIASHTTEERGLNVTLSSTGRNGFKSHALQLNNRQ
IRGLEEELQFSLGSKINVKVGGNSKGTLKVLRTYNVLDMKNTTCQDLQIE
VTVKGHVEYTMEANEDYEDYEYDELPAKDDPDAPLQPVTPLQLFEGRRNR
RRREAPKVVEEQESRVHYTVCIWRNGKVGLSGMAIADVTLLSGFHALRAD
LEKLTSLSDRYVSHFETEGPHVLLYFDSVPTSRECVGFEAVQEVPVGLVQ
PASATLYDYYNPERRCSVFYGAPSKSRLLATLCSAEVCQCAEGKCPRQRR
ALERGLQDEDGYRMKFACYYPRVEYGFQVKVLREDSRAAFRLFETKITQV
LHFTKDVKAAANQMRNFLVRASCRLRLEPGKEYLIMGLDGATYDLEGHPQ
YLLDSNSWIEEMPSERLCRSTRQRAACAQLNDFLQEYGTQGCQV

SEQ ID NO: 3 (residues of rituximab heavy chain up to hinge)

AVYYCARSTYYGGDWYFNVWGAGTTVTVSAASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSCDKTHTCPP

SEQ ID NO: 4

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGTGATTGGTACTTCGACGTCTGGGGAGCCGGAACCACTGT
GACCGTGTCAGCCGCATCCACCAAGGGCCCATCGGTCTTCTCCCTGGCAC
CCTCCTACAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAA

SEQ ID NO: 5 amino acid changes of N to D at codon 109, P to S at codon 131, and S to Y at codon 136

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFDVWGAGTTVTVSAASTKGPSVFSLAPSYKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 6

QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

SEQ ID NO: 7

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCGACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 8 amino acid change of N to D at codon 109

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFDVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 9

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCTCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 10 amino acid change of P to S at codon 131

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFSLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 11

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCCCCCTGGCAC
CCTCCTACAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 12 amino acid change of S to Y at codon 136

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSYKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 13

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCGACGTCTGGGGAGCCGGAACCACTGT
GACCGTGTCAGCCGCATCCACCAAGGGCCCATCGGTCTTCTCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 14 amino acid changes of N to D at codon 109, and P to S at codon 131

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFDVWGAGTTVTVSAASTKGPSVFSLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 15

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCGACGTCTGGGGAGCCGGAACCACTGT
GACCGTGTCAGCCGCATCCACCAAGGGCCCATCGGTCTTCCCCCTGGCAC
CCTCCTACAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 16 amino acid changes of N to D at codon 109, and S to Y at codon 136

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFDVWGAGTTVTVSAASTKGPSVFPLAPSYKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 17

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCTCCCTGGCAC
CCTCCTACAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 18 amino acid changes of P to S at 131 and S to Y at 136

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFSLAPSYKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 19

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTGCTTCAACGTCTGGGGAGCCGGAACCACTGT
GACCGTGTCAGCCACATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAA

SEQ ID NO: 20 amino acid changes of Y to C at codon 107, and A to T at codon 122

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWCFNVWGAGTTVTVSATSTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 21

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTGCTTCAACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 22 amino acid changes of Y to C at codon 107

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWCFNVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 23

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCACATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 24 amino acid changes of A to T at codon 122

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSATSTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 25

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTATGGCGGCGATTGGTACTTAAACGTCTGGGGAGCCGGAACCACTGT
GACCGTGTCAGCCGCATCCACCAAGGGCCCATCGGTCTACCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAA

SEQ ID NO: 26 amino acid changes of F to L at codon 108, and F to Y at codon 130

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYLNVWGAGTTVTVSAASTKGPSVYPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 27

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTAAACGTCTGGGGAGCCGGAACCACTGT
GACCGTGTCAGCCGCATCCACCAAGGGCCCATCGGCTTCCCCCTGGCACC
CTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTG
ACCAGCGGCGTGCACACCTTCCCGGCTGTCCTACAGTCCTCAGGACTCTA
CTCCCTCAGCAGCGTGGTGACCGTGCCCTCCAGCAGCTTGGGCACCCAGA
CCTACATCTGCAACGTGAATCACAAGCCCAGCAACACCAAGGTGGACAAG
AAAGTTGAGCCCAAATCTTGTGACAAAACTCACACATGCCCACCGTGCCC
AGCACCTGAACTCCTGGGGGGACCGTCAGTCTTCCTCTTCCCCCCAAAAC
CCAAGGACACCCTCATGATCTCCCGGACCCCTGAGGTCACATGCGTGGTG
GTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGC
CCCCATCGAGAAAACCATCTCCAAAGCCAAAGGGCAGCCCCGAGAACCAC
AGGTGTACACCCTGCCCCCATCCCGGGATGAGCTGACCAAGAACCAGGTC
AGCCTGACCTGCCTGGTCAAAGGCTTCTATCCCAGCGACATCGCCGTGGA
GTGGGAGAGCAATGGGCAGCCGGAGAACAACTACAAGACCACGCCTCCCG
TGCTGGACTCCGACGGCTCCTTCTTCCTCTATAGCAAGCTCACCGTGGAC
AAGAGCAGGTGGCAGCAGGGGAACGTCTTCTCATGCTCCGTGATGCATGA
GGCTCTGCACAACCACTACACGCAGAAGAGCCTCTCCCTGTCTCCCGGGA
AA

SEQ ID NO: 28 amino acid changes of F to L at codon 108

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYLNVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 29

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTACCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 30 amino acid changes of F to Y at codon 130

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVYPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 31

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACTGCGGCGATTGGTACTTCAACGTCTGGGGAGCCGGAACCACTGT
GACCGTGTCAGCCGCATCCACCAAGGGCCCATCGGTCTTCCCCCTGGCAT
CCTCCTCCAAGAGCACCTCTGGGGGCACAGCGGCCCTGGGCTGCCTGGTA
AAGGACTACTTCCCCGAACCGGTGACGGTGTCGTGGAACTCAGGCGCCCT
GACCACCGGCGTGCACACCTTCCCGGCTGTCCTACAGTCCTCAGGACTCT
ACTCCCTCAGCAGCGTGGTGACCGTGCCCTCCAGCCGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAA

SEQ ID NO: 32 amino acid changes of G to C at codon 103, P to S at codon 134, S to T at codon 169, and S to R at codon 196

QVQLQQPGAELVKPGASVKMSCKASGYTFTSYNMHWVKQTPGRGLEWIG
AIYPGNGDTSYNQKFKGKATLTADKSSSTAYMQLSSLTSEDSAVYYCAR
STYYCGDWYFNVWGAGTTVTVSAASTKGPSVFPLASSSKSTSGGTAALGC
LVKDYFPEPVTVSWNSGALTTGVHTFPAVLQSSGLYSLSSWTVPSSRLGT
QTYICNVNHKPSNTKVDKKVEPKSCDKTHTCPPCPAPELLGGPSVFLFPP
KPKDTLMISRTPEVTCVWDVSHEDPEVKFNWYVDGVEVHNAKTKPREEQY
NSTYRWSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

SEQ ID NO: 33

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACTGCGGCGATTGGTACTTCAACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 34 amino acid changes of G to C at codon 103

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYCGDWYFNVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 35

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCCCCCTGGCAT
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 36 amino acid changes of P to S at codon 134

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLASSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 37

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTTCGGCGGCGATTGGTACTTCAACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 38 amino acid changes of Y to F at codon 102

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YFGGDWYFNVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 39

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCA
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

SEQ ID NO: 40 amino acid changes of L to Q at codon 167

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGAQTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVWDVSHEDPEVKFNWYVDGVEVHNAKTKPREEQYN
STYRWSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

SEQ ID NO: 41 (CDRH1)

GYTFTSYN

SEQ ID NO: 42 (CDRH2)

IYPGNGDT

SEQ ID NO: 43 (CDRH3)

ARSTYYGGDWYFNV

SEQ ID NO: 44 (CDRL1)

SSSVSY

SEQ ID NO: 45 (CDRL2)

ATS

SEQ ID NO: 46 (CDRL3 )

QQWTSNPPT

SEQ ID NO: 47 (rituximab heavy chain wild type)

QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSWTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

SEQ ID NO: 48 forward primer for random mutagenesis PCR

CGCCGTGTATTACTGTGCT

SEQ ID NO: 49 reverse primer for random mutagenesis PCR

GGTGGGCATGTGTGAGTTT

SEQ ID NO: 50 (nucleic acid encoding rituximab heavy chain wild type)

CAAGTGCAGCTGCAGCAGCCGGGTGCAGAACTCGTGAAGCCAGGGGCCTC
AGTGAAGATGTCCTGCAAAGCCAGCGGCTACACCTTCACCTCCTACAACA
TGCACTGGGTCAAGCAAACTCCTGGACGCGGACTTGAGTGGATTGGTGCT
ATCTACCCCGGAAACGGCGACACCAGCTACAATCAGAAGTTTAAGGGGAA
GGCCACTCTGACTGCCGACAAGTCGTCCTCGACGGCGTACATGCAGCTGA
GCTCGCTGACCTCCGAGGACTCCGCCGTGTATTACTGTGCTCGGTCCACC
TACTACGGCGGCGATTGGTACTTCAACGTCTGGGGAGCCGGAACCACTGT
GACCGTGTCAGCCGCATCCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAAAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGATGAGCTGACCAAGAACCAGGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCCGGG
AAA

We claim:

1. An immuno-suppression refractory protein or polypeptide comprising the amino acid residue sequence of SEQ ID NO: 3, wherein one, two, or three amino acid residues in said sequence are substituted with an amino acid residue different than shown in SEQ ID NO: 3, wherein the one, two, or three substituted amino acid residues reduce or eliminate binding of the immuno-suppression refractory protein or polypeptide to an immuno-suppressive protein, relative to the immuno-suppression refractory protein or polypeptide without the one, two, or three amino acid substitutions.

2. The immuno-suppression refractory protein or polypeptide of claim 1 which is a full-length antibody.

3. The immuno-suppression refractory protein or polypeptide of claim 1 which is a bispecific antibody.

4. The immuno-suppression refractory protein or polypeptide of claim 1 wherein the immuno-suppressive protein is CA125/MUC16.

5. The immuno-suppression refractory protein or polypeptide of claim 1 selected from the group consisting of:
a. an antibody containing a light chain according to SEQ ID NO: 6 and a heavy chain comprising SEQ ID: 5;
b. an antibody containing a light chain according to SEQ ID NO: 6 and a heavy chain comprising SEQ ID: 8;
c. T an antibody containing a light chain according to SEQ ID NO: 6 and a heavy chain comprising SEQ ID: 10;
d. an antibody containing a light chain according to SEQ ID NO: 6 and a heavy chain comprising SEQ ID: 12;
e. an antibody containing a light chain according to SEQ ID NO: 6 and a heavy chain comprising SEQ ID: 14;
f. comprises an antibody containing a light chain according to SEQ ID NO: 6 and a heavy chain comprising SEQ ID: 16;
g. an antibody containing a light chain according to SEQ ID NO: 6 and a heavy chain comprising SEQ ID: 18;
h. an antibody containing a light chain according to SEQ ID NO: 6 and a heavy chain comprising SEQ ID: 20;
i. an antibody containing a light chain according to SEQ ID NO: 6 and a heavy chain comprising SEQ ID: 22;
j. an antibody containing a light chain according to SEQ ID NO: 6 and a heavy chain comprising SEQ ID: 24;
k. an antibody containing a light chain according to SEQ ID NO: 6 and a heavy chain comprising SEQ ID: 26;
1. an antibody containing a light chain according to SEQ ID NO: 6 and a heavy chain comprising SEQ ID: 28;
m. an antibody containing a light chain according to SEQ ID NO: 6 and a heavy chain comprising SEQ ID: 30;
n. an antibody containing a light chain according to SEQ ID NO: 6 and a heavy chain comprising SEQ ID: 32;
o. an antibody containing a light chain according to SEQ ID NO: 6 and a heavy chain comprising SEQ ID: 34;
p. an antibody containing a light chain according to SEQ ID NO: 6 and a heavy chain comprising SEQ ID: 36;
q. an antibody containing a light chain according to SEQ ID NO: 6 and a heavy chain comprising SEQ ID: 38; and
r. an antibody containing a light chain according to SEQ ID NO: 6 and a heavy chain comprising SEQ ID: 40.

6. The immuno-suppression refractory protein or polypeptide of claim 1 which is a protein that consists of an immunoglobulin light chain and an immunoglobulin heavy chain of an IgG isotype antibody.

7. The immuno-suppression refractory protein or polypeptide of claim 1 which is a full length IgG isotype antibody.

8. The immuno-suppression refractory protein or polypeptide of claim 1 which is an antigen-binding fragment or Fab domain of an IgG isotype antibody.

9. The immuno-suppression refractory protein or polypeptide of claim 1 wherein the amino acid residues are selected from positions 102, 103, 107, 108, 109, 122, 130, 131, 134, 136, 151, 161, 167, 169, 189, 196, 201, and 214 of rituximab IgG1 heavy chain as shown in SEQ ID NO: 47.

10. The immuno-suppression refractory protein or polypeptide of claim 1 which binds to CD20 with an affinity/Kd that is > 1 nM or < 400 nM.

11. The immuno-suppression refractory protein or polypeptide of claim 1 which binds to CD20 with an affinity/Kd that is > 10 nM or < 50 nM.

12. The immuno-suppression refractory protein or polypeptide of claim 1 which binds to CD20 with an affinity that is ± 50% of the affinity/Kd of the immuno-suppression refractory protein or polypeptide comprising the amino acid residue sequence of SEQ ID NO: 3 for CD20.

13. The immuno-suppression refractory protein or polypeptide of claim 1 wherein the amino acid residues are selected from positions in region CDR3 of rituximab heavy chain as shown in SEQ ID NO: 47.

14. The immune-suppression refractory protein or polypeptide of claim 1 wherein the amino acid residues are selected from positions in framework region 4 of rituximab as shown in SEQ ID NO: 47.

15. A nucleic acid vector encoding the immuno-suppression refractory protein or polypeptide of claim 1.

16. A polynucleotide encoding the immuno-suppression refractory protein or polypeptide of claim 1.

17. A stable cell line comprising the nucleic acid vector of claim 15 and expressing the immuno-suppression refractory protein or polypeptide.

18. A method to treat a patient with a disease who expresses an elevated level of CA125 compared to a population of healthy humans, comprising:
administering to the patient the immuno-suppression refractory protein or polypeptide of claim 1.

19. The method of claim 18 wherein the disease is cancer.

20. The method of claim 18 wherein the disease is an inflammatory disease.

21. The method of claim 19 wherein the cancer is selected from the group consisting of Hodgkin’s Lymphoma, Non-Hodgkin’s Lymphoma, Follicular Lymphoma, Large Cell Lymphoma, Diffuse Large B-cell Lymphoma, Chronic Lymphocytic Leukemia, and multiple myeloma.

22. A method of treating a cancer patient or a patient with an inflammatory disease, comprising:
administering to the cancer patient or the patient with an inflammatory disease a polypeptide selected from the group consisting of: (a) a full-length human complement proprotein C3B or C4B,
(b) a naturally occurring, proteolytic fragment of human complement proprotein C3B or C4B that is capable of binding IgG, or
(c) a portion of human complement proprotein C3B or C4B that is capable of binding IgG.

23. The method of claim 22 wherein the polypeptide comprises amino acid sequences shown in SEQ ID NO: 1 or SEQ ID NO: 2.

24. The method of claim 22 wherein the cancer patient or the patient with inflammatory disease expresses elevated CA125 levels compared with a control, normal human population.

25. The method of claim 22 wherein the polypeptide is administered in combination with an anti-CD20 antibody.

26. The method of claim 25 wherein the anti-CD20 antibody is rituximab.

27. The method of claim 22 wherein the patient is a cancer patient, and further comprising:
administering to the cancer patient an antibody that targets a tumor, wherein humoral immune responses mediated by the antibody are suppressed by CA125 in the absence of the polypeptide.

28. The method of claim 22 wherein the patient is a cancer patient and the patient has a cancer selected from the group consisting of Hodgkin’s Lymphoma, Non-Hodgkin’s Lymphoma, Follicular Lymphoma, Large Cell Lymphoma, Diffuse Large B-cell Lymphoma, Chronic Lymphocytic Leukemia, and multiple myeloma.

29. A combination comprising (a) an anti-CD20 antibody and (b) full-length complement proprotein C3B or C4B, a naturally occurring, proteolytic fragment of complement proprotein C3B or C4B that is capable of binding IgG, or a portion of complement proprotein C3B or C4B that is capable of binding IgG.

30. The combination of claim 29 wherein the anti-CD20 antibody is rituximab.

31. The combination of claim 29 which comprises the naturally occurring, proteolytic fragment of complement proprotein C3B or C4B, wherein the naturally occurring, proteolytic fragment of complement proprotein C3B or C4B is selected from the group consisting of C3B residues 23-1663, C3B residues 23-667, C3B residues 569-667, C3B residues 672-1663, C3B residues 672-748, C3B residues 672-747, C3B residues 749-1663, C3B residues 749-954, C3B residues 955-1303, C3B residues 955-1001, C3B residues 1002-1303, C3B residues 1304-1320, C3B residues 1321-1663, C4B residues 20-675, C4B residues 676-679, C4B residues 680-1446, C4B residues 680-756, C4B residues 757-1446, C4B residues 957-1336, C4B residues 1447-1453, and C4B residues 1454-1744.

32. A human cancer cell line that expresses human proteins CA125 and CD20.

33. The human cancer cell line of claim 32 that is a human ovarian cancer cell line.

34. The human cancer cell line of claim 33 which is made by transducing cells of cell line OVCAR3 with an expression vector encoding CD20.

35. A method of screening candidate antibodies that bind to CD20, comprising:
a. contacting a candidate antibody with the human cancer cell line of claim 36; and
b. determining antibody dependent cellular cytotoxicity (ADCC) or complement dependent cytotoxicity (CDC) of the human cancer cell line initiated by the candidate antibody.

36. A method for testing and treating a tumor expressing CA125 in a patient, comprising:
contacting (a) a body fluid sample isolated from the patient; with
(b) an antibody comprising the amino acid sequence of SEQ ID NO: 3; and contacting (a) with (c) an immuno-suppression refractory protein or polypeptide comprising the amino acid residue sequence of SEQ ID NO: 3, wherein one, two, or three amino acid residues in said sequence are substituted with an amino acid residue different than shown in SEQ ID NO: 3, wherein the one, two, or three substituted amino acid residues reduce or eliminate binding of the immuno-suppression refractory protein or polypeptide to an immuno-suppressive protein, relative to the immuno-suppression refractory protein or polypeptide without the one, two, or three amino acid substitutions; and
determining that CA125 in the body fluid sample binds to (b) but not (c) and treating the patient with (c); or
determining that CA125 in the body fluid sample binds to (b) and/or (c) and treating the patient with full-length complement proprotein C3B or C4B, a naturally occurring, proteolytic fragment of complement proprotein C3B or C4B that is capable of binding IgG, or a portion of complement proprotein C3B or C4B that is capable of binding IgG.

37. The method of claim 22 or 36 wherein the naturally occurring, proteolytic fragment of complement proprotein C3B or C4B is administered, wherein the naturally occurring, proteolytic fragment of complement protein C3B or C4B is selected from the group consisting of C3B residues 23-1663, C3B residues 23-667, C3B residues 569-667, C3B residues 672-1663, C3B residues 672-748, C3B residues 672-747, C3B residues 749-1663, C3B residues 749-954, C3B residues 955-1303, C3B residues 955-1001, C3B residues 1002-1303, C3B residues 1304-1320, C3B residues 1321-1663, C4B residues 20-675, C4B residues 676-679, C4B residues 680-1446, C4B residues 680-756, C4B residues 757-1446, C4B residues 957-1336, C4B residues 1447-1453, C4B residues 1454-1744.


SEQUENCE LISTING (50 sequences)

SEQ ID NO: 1 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 2 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 3 - protein, 141 aa, Artificial Sequence
  rituximab heavy chain wild type
AVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 4 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggtgattggtacttcgacgtctggggagccggaaccactgtgaccgtgtcagccgcatccaccaagggcccatcggtcttctccctggcaccctcctacaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 5 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGAGTTVTVSAASTKGPSVFSLAPSYKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 213 aa, Artificial Sequence
  mutant heavy chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcgacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 8 - protein, 451 aa, Artificial Sequence
  encoding mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttctccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 10 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFSLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttccccctggcaccctcctacaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 12 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSYKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcgacgtctggggagccggaaccactgtgaccgtgtcagccgcatccaccaagggcccatcggtcttctccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 14 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGAGTTVTVSAASTKGPSVFSLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcgacgtctggggagccggaaccactgtgaccgtgtcagccgcatccaccaagggcccatcggtcttccccctggcaccctcctacaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 16 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGAGTTVTVSAASTKGPSVFPLAPSYKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttctccctggcaccctcctacaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 18 - protein, 451 aa, Artificial Sequence
  mutant heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFSLAPSYKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtgcttcaacgtctggggagccggaaccactgtgaccgtgtcagccacatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWCFNVWGAGTTVTVSATSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtgcttcaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 22 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWCFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccacatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 24 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSATSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactatggcggcgattggtacttaaacgtctggggagccggaaccactgtgaccgtgtcagccgcatccaccaagggcccatcggtctaccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYLNVWGAGTTVTVSAASTKGPSVYPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttaaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 28 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYLNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtctaccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 30 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVYPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactactgcggcgattggtacttcaacgtctggggagccggaaccactgtgaccgtgtcagccgcatccaccaagggcccatcggtcttccccctggcatcctcctccaagagcacctctgggggcacagcggccctgggctgcctggtaaaggactacttccccgaaccggtgacggtgtcgtggaactcaggcgccctgaccaccggcgtgcacaccttcccggctgtcctacagtcctcaggactctactccctcagcagcgtggtgaccgtgccctccagccgcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYCGDWYFNVWGAGTTVTVSAASTKGPSVFPLASSSKSTSGGTAALGCLVKDYFPEPVTVSWNSGALTTGVHTFPAVLQSSGLYSLSSVVTVPSSRLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactactgcggcgattggtacttcaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 34 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYCGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttccccctggcatcctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 36 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLASSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctacttcggcggcgattggtacttcaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 38 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYFGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1353 bp, Artificial Sequence
  encoding mutant heavy chain
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgcccagaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 40 - protein, 451 aa, Artificial Sequence
  heavy chain mutant
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGAQTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 8 aa, Homo sapiens
  PEPTIDE (0)...(0): CDRH1
GYTFTSYN

SEQ ID NO: 42 - protein, 8 aa, Homo sapiens
  DOMAIN (0)...(0): CDRH2
IYPGNGDT

SEQ ID NO: 43 - protein, 14 aa, Homo sapiens
  DOMAIN (0)...(0): CDRH3
ARSTYYGGDWYFNV

SEQ ID NO: 44 - protein, 6 aa, Homo sapiens
  DOMAIN (0)...(0): CDRL1
SSSVSY

SEQ ID NO: 45 - protein, 3 aa, Homo sapiens
  DOMAIN (0)...(0): CDRL2
ATS

SEQ ID NO: 46 - protein, 9 aa, Homo sapiens
  DOMAIN (0)...(0): CDRL3
QQWTSNPPT

SEQ ID NO: 47 - protein, 451 aa, Artificial Sequence
  rituximab heavy chain wild type
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 19 bp, Homo sapiens
cgccgtgtattactgtgct

SEQ ID NO: 49 - DNA, 19 bp, Homo sapiens
ggtgggcatgtgtgagttt

SEQ ID NO: 50 - DNA, 1353 bp, Homo sapiens
caagtgcagctgcagcagccgggtgcagaactcgtgaagccaggggcctcagtgaagatgtcctgcaaagccagcggctacaccttcacctcctacaacatgcactgggtcaagcaaactcctggacgcggacttgagtggattggtgctatctaccccggaaacggcgacaccagctacaatcagaagtttaaggggaaggccactctgactgccgacaagtcgtcctcgacggcgtacatgcagctgagctcgctgacctccgaggactccgccgtgtattactgtgctcggtccacctactacggcggcgattggtacttcaacgtctggggagccggaaccactgtgaccgtgtcagccgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcccgggaaa